(19) United States
(12) Patent Application Publication
(10) Pub. No.: US 2023/0227514 A1
(43) Pub. Date: Jul. 20, 2023

(54) BETA-ARRESTIN COMPOSITIONS AND ASSOCIATED METHODS

(71) Applicant: Duke University, Durham, NC (US)

(72) Inventor: Sudha SHENOY, Durham, NC (US)

(73) Assignee: Duke University, Durham, NC (US)

(21) Appl. No.: 17/914,598

(22) PCT Filed: Mar. 25, 2021
(86) PCT No.: PCT/US2021/024178

§ 371 Date: Sep. 26, 2022

Related U.S. Application Data
(60) Provisional application No. 63/000,075, filed on Mar. 26, 2020.

(51) Int. Cl. C07K 14/47; G01N 33/50

(52) U.S. Cl. CPC C07K 14/4702; G01N 33/5035; C07K 2319/60; C07K 2319/95; G01N 2333/726

(57) ABSTRACT

Provided herein are compositions and methods for assessing arrestin-dependent signaling. The provided compositions include fusion proteins comprising arrestin polypeptides that bind strongly to G protein-coupled receptors (GPCRs). In some instances, the fusion proteins may also bind to non-GPCR proteins, such as single transmembrane receptors and non-receptor proteins. Also provided are nucleic acids, vectors, constructs, and host cells that encode or express such fusion proteins. Also provided are methods of using such fusion proteins to assess arrestin trafficking, localization, and other functions including, for example, arrestin-mediated GPCR signaling as well as non-GPCR protein activity or signaling.

### CROSS-REFERENCE TO RELATED APPLICATIONS

This application claims the benefit of U.S. Provisional Application No. 63/000,075, filed Mar. 26, 2020. This provisional application is incorporated by reference herein in its entirety for all purposes.

### SEQUENCE LISTING

The official copy of the sequence listing is submitted electronically via EFS-Web as an ASCII formatted sequence listing with a file named 1240538_seqlist.txt, created on Mar. 22, 2021, and having a size of 33.7 KB, and is filed concurrently with the specification. The sequence listing contained in this ASCII formatted document is part of the specification and is herein incorporated by reference in its entirety.

### BACKGROUND

G protein-coupled receptors (GPCRs) are members of a large family of cell surface receptors. GPCRs are activated by a wide variety of stimulants (or “agonists”), including light, odorant molecules, peptide and non-peptide neurotransmitters, hormones, growth factors and lipids, and control a wide variety of physiological processes including sensory transduction, cell-cell communication, neuronal transmission, and hormonal signaling. Through interaction with G proteins, GPCRs regulate many downstream processes via mechanisms including protein phosphorylation, regulation of translation, and regulation of transcription. Arrestin proteins are a small family of proteins important for regulating GPCR signaling, both through uncoupling of GPCRs from G proteins (i.e., receptor desensitization) and through alternative, G protein-independent GPCR signaling pathways. In addition to GPCRs, arrestins can bind other types of cell-surface receptors, ion channels, and engage many signaling and biochemical pathways.

Given their importance in health and disease, together with their potential for therapeutic intervention via using small molecules as regulators, G protein-coupled receptors represent the largest family of druggable targets. GPCR assay development and GPCR ligand screening are a major focus of drug discovery research worldwide. There is a strong desire for drugs that specifically target GPCR signaling. As such, there is a need for assays to study the various mechanisms by which GPCRs are regulated and for methods to identify drugs that impact these mechanisms.

### BRIEF SUMMARY

The Brief Summary is provided to introduce a selection of concepts that are further described below in the Detailed Description. This Brief Summary is not intended to identify key or essential features of the claimed subject matter, nor is it intended to be used as an aid in limiting the scope of the claimed subject matter.

Provided herein are fusion proteins that can be used to assess arrestin-dependent signaling. In some embodiments, the fusion proteins comprise an arrestin polypeptide fused to a ubiquitin-like protein (UBL). In some embodiments, the arrestin polypeptide is fused to the UBL protein via a peptide linker. In some embodiments, the arrestin polypeptide comprises an amino acid sequence having at least 80% identity to SEQ ID NO:3, SEQ ID NO:4, SEQ ID NO:5, or SEQ ID NO:6. In some embodiments, the UBL comprises an amino acid sequence having at least 80% identity to SEQ ID NO:7, SEQ ID NO:8, SEQ ID NO:9, SEQ ID NO:10, SEQ ID NO:11, SEQ ID NO:12, or SEQ ID NO:13. In some embodiments, the fusion protein comprises an amino acid sequence having at least 80% identity to SEQ ID NO:1 or SEQ ID NO:2.

In some embodiments, the fusion protein further comprises a detectable moiety. In some embodiments, the detectable moiety is a fluorescent moiety. In some embodiments, the fusion protein is resistant to de-SUMOylation.

In some embodiments, the fusion protein displays increased binding to a G protein-coupled receptor (GPCR) upon expression in cells, wherein the increased binding is measured relative to the wild-type form of the arrestin polypeptide.

Also provided is a recombinant nucleic acid encoding any of the fusion proteins described herein. Further provided is a DNA construct comprising a promoter operably linked to the recombinant nucleic acid. In some embodiments, the promoter is an inducible promoter or a constitutive promoter. Also provided is a vector comprising a recombinant nucleic acid described herein. Also provided is a vector comprising a vector comprising a DNA construct described herein. Also provided is a host cell comprising a recombinant nucleic acid as described herein, a host cell comprising a DNA construct as described herein, and a host cell comprising a vector described herein. In some embodiments, the host cell is a mammalian cell.

Also provided is a cell population comprising a plurality of cells. In some embodiments, the plurality of cells comprise a recombinant nucleic acid described herein. In some embodiments, the plurality of cells comprise a DNA construct described herein. In some embodiments, the plurality of cells comprise a vector described herein. In some embodiments, the plurality of cells comprise a plurality of the host cells described herein. In some embodiments, the plurality of cells express any of the fusion proteins described herein. In some embodiments, the plurality of cells expresses the fusion protein stably or transiently. In some embodiments, the plurality of cells expresses the fusion protein inducibly or constitutively.

Also provided is a method for detecting a protein subcellular localization pattern, the method comprising: (a) providing a plurality of cells that express a fusion protein described herein; and (b) detecting the subcellular localization pattern of the fusion protein in the plurality of cells. In some embodiments, the plurality of cells also express a G protein-coupled receptor (GPCR) and/or a non-GPCR protein, and the method further comprises detecting the subcellular localization pattern of the GPCR and/or the non-GPCR protein in the plurality of cells. In some embodiments, the plurality of cells is treated with an agonist compound that activates the GPCR and/or the non-GPCR protein prior to detecting the subcellular localization pattern of the fusion protein, the GPCR, and/or the non-GPCR protein in the plurality of cells.

Also provided is a method for detecting protein-protein interaction of an arrestin protein and a G protein-coupled receptor (GPCR), the method comprising: (a) providing a plurality of cells that express a fusion protein described herein and a GPCR; and (b) detecting the protein-protein interaction of the fusion protein with the GPCR in the plurality of cells. In some embodiments, the plurality of cells is treated with an agonist compound that activates the GPCR prior to detecting the protein-protein interaction of the fusion protein with the GPCR.

Also provided is a method for identifying whether a drug compound impacts arrestin-mediated G protein-coupled receptor (GPCR) signaling, the method comprising: (a) providing a plurality of cells that express a fusion protein described herein and a GPCR, wherein the fusion protein is able to bind to and regulate the signaling of the GPCR; (b) treating the plurality of cells with a drug compound, thereby forming a drug-treated plurality of cells; (c) assessing activation and/or signaling of the GPCR in the drug-treated plurality of cells; and (d) comparing the GPCR activation and/or signaling in the drug-treated plurality of cells to the GPCR activation and/or signaling assessed in a control plurality of cells that have not been contacted with the drug compound, wherein a difference in GPCR activation and/or signaling between the drug-treated plurality of cells and the control plurality of cells indicates that the drug compound impacts arrestin-mediated GPCR signaling of the GPCR. In some embodiments, the drug-treated plurality of cells and the control plurality of cells are treated with an agonist compound that activates the GPCR prior to assessing activation and/or signaling of the GPCR in the drug-treated plurality of cells and in the control plurality of cells. In some embodiments, assessing activation and/or signaling of the GPCR comprises detecting the subcellular localization pattern of the fusion protein. In some embodiments, assessing activation and/or signaling of the GPCR comprises detecting protein-protein interaction of the fusion protein and the GPCR.

In some embodiments, in any of the methods described herein, the GPCR may be angiotensin type 1a receptor (AT₁ₐR), β₂ adrenergic receptor (β₂AR), D₂ dopamine receptor (D2R), β₁ adrenergic receptor (β₁AR), D₁ dopamine receptor (D1R), V₂ vasopressin receptor (V₂R), and/or glucagon receptor (GCGR).

Also provided is a method for detecting protein-protein interaction of an arrestin protein and a non-GPCR protein, the method comprising: (a) providing a plurality of cells that express a fusion protein described herein and a non-GPCR protein; and (b) detecting the protein-protein interaction of the fusion protein with the non-GPCR protein in the plurality of cells. In some embodiments, the plurality of cells is treated with an agonist compound that activates the non-GPCR protein prior to detecting the protein-protein interaction of the fusion protein with the non-GPCR protein.

Also provided is a method for identifying whether a drug compound impacts arrestin-mediated signaling or activity, the method comprising: (a) providing a plurality of cells that express a fusion protein described herein and a non-GPCR signaling protein, wherein the fusion protein is able to bind to and regulate the signaling of the non-GPCR signaling protein; (b) treating the plurality of cells with a drug compound, thereby forming a drug-treated plurality of cells; (c) assessing activation and/or signaling of the non-GPCR signaling protein in the drug-treated plurality of cells; and (d) comparing the non-GPCR signaling protein activation and/or signaling in the drug-treated plurality of cells to the non-GPCR signaling protein activation and/or signaling assessed in a control plurality of cells that have not been contacted with the drug compound, wherein a difference in non-GPCR signaling protein activation and/or signaling between the drug-treated plurality of cells and the control plurality of cells indicates that the drug compound impacts arrestin-mediated signaling or activity. In some embodiments, the drug-treated plurality of cells and the control plurality of cells are treated with an agonist compound that activates the non-GPCR signaling protein prior to assessing activation and/or signaling of the non-GPCR signaling protein in the drug-treated plurality of cells and in the control plurality of cells. In some embodiments, assessing activation and/or signaling of the non-GPCR signaling protein comprises detecting the subcellular localization pattern of the fusion protein. In some embodiments, assessing activation and/or signaling of the non-GPCR signaling protein comprises detecting protein-protein interaction of the fusion protein and the non-GPCR signaling protein.

In some embodiments, in any of the methods described herein, the non-GPCR protein may be a single transmembrane protein. In some embodiments, the non-GPCR protein may be one or more of insulin-like growth factor 1 receptor (IGF1-R), a transforming growth factor beta receptor (TGF-R), a Notch receptor, a receptor tyrosine kinase (e.g., an insulin receptor), an interleukin receptor, or a toll-like receptor. In some embodiments, in any of the methods described herein, the non-GPCR protein may be a non-receptor protein. In some embodiments, the non-receptor protein may be an endocytic protein. In some embodiments, the endocytic protein may be a ras-related nuclear protein (Ran) or a member of the Rab protein family. In some embodiments, the non-receptor protein may be a protein that localizes to the nuclear membrane. In some embodiments, the non-receptor protein may be RanGAP1. In some embodiments, the non-receptor protein may be a mitogen-activated protein kinase. In some embodiments, the mitogen-activated protein kinase may be one or more of an extracellular signal-regulated kinase (ERK), a p38 mitogen-activated protein kinase, or a c-Jun N-terminal kinase (JNK). In some embodiments, the non-receptor protein may be tumor protein P53 (p53). In some embodiments, the non-receptor protein may be mouse double minute 2 homolog (MDM2).

In some embodiments, in any of the methods described herein, detecting the subcellular localization pattern of the fusion protein or the interaction of the fusion protein with the GPCR and/or the non-GPCR protein may be performed by immunostaining, confocal microscopy, bioluminescence resonance energy transfer (BRET), affinity chromatography, and/or immunoprecipitation. In some embodiments, the fusion protein comprises a fluorescent moiety and the GPCR and/or the non-GPCR protein comprises a luciferase tag.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1 shows that SUMOylation at the canonical site in beta-arrestin-2 (βarrestin2) is not required for association with agonist-activated GPCRs, according to certain aspects of this disclosure. HEK-293 cells were transfected with vector, FLAG-βarrestin2 (WT) or FLAG-βarrestin2-K296R along with His6-SUMO1. FLAG immunoprecipitates were serially probed for His6-SUMO1 and βarrestin2 (left panel). Lysates were analyzed for SUMO1, βarrestin2 and GAPDH as shown. The bar graph (right panel) shows the means±SEM for the ratios obtained by dividing SUMO1 bands by the cognate βarrestin2 bands detected in the IP samples in the left panel, and the data are summarized from three independent experiments. **p=0.006 versus WT, unpaired t test.

FIG. 2 shows that a βarrestin2-SUMO1 fusion protein displays stronger association with agonist-activated β₂AR than βarrestin2, according to certain aspects of this disclosure. HEK-293 cells stably expressing a Flag-β₂AR were transiently transfected with either vector, YFP-βarrrestin2-WT or YFP-βarrestin2-SUMO1 and were exposed (or not exposed) to the β₂AR agonist Isoproterenol (Iso, 1 μM) for the indicated times. Flag-tagged receptors were immunoprecipitated after chemical cross-linking and the IP was probed with an anti-GFP antibody that recognizes YFP (MBL, International) and subsequently blots were stripped and reprobed with a β₂AR specific antibody, H-20 (top panel). The bar graph (bottom panel) shows immunoreactivities of the different βarrrestin2 fusion proteins recovered normalized to the cognate amount of receptors and expressed as ratios of relative intensities of the labeled bands. Statistical comparison was done using two-way ANOVA and shown as means±SEM of 4 independent experiments. #p<0.05 versus respective unstimulated condition; *p<0.05 versus all other conditions, two-way ANOVA, Holm-Sidak's comparison.

FIG. 3 shows BRET analysis of protein-protein interactions between agonist-activated β₂AR and βarrestin2, βarrestin2-SUMO1, and βarrestin2-Ub as a measure of recruitment to the β₂AR, according to certain aspects of this disclosure. HEK-293T cells were transiently transfected with a fixed amount of β₂AR-Rluc and increasing amounts of YFP-βarrestin2-WT (top panel), YFP-βarrestin2-SUMO1 (middle panel) or YFP-βarrestin2-Ub (bottom panel). Cells were stimulated (squares) or not stimulated (circles) with Iso (1 μM) for 5 min.

FIG. 4 shows agonist-induced BRET changes (top panel) that correspond to the pure agonist effect, according to certain aspects of this disclosure. The pure agonist effect was determined by subtracting the vehicle curve from the Iso-stimulated curve of the data shown in FIG. 3. The histogram (bottom panel) represents BRET max (Bmax) values generated from the agonist-induced BRET change curves, according to certain aspects of this disclosure. Significance of agonist-induced changes was established by one-way ANOVA comparing signals generated by YFP-βarr2-WT to those generated by YFP-βarr2-SUMO1 or YFP-βarr2-Ub cells *p<0.05; **p<0.01. Results correspond to mean±SEM of 3 independent experiments performed in duplicates.

FIG. 5 shows that a βarrestin2-SUMO1 fusion protein displays stronger association with agonist-activated D₂ dopamine receptor (D2R) than βarrestin2 (βarr2), according to certain aspects of this disclosure. HEK-293 cells stably expressing the HA-D2R were transiently transfected with either vector, YFP-βarr2-WT or YFP-βarr2-SUMO1 and were exposed or not to dopamine (DA, 1 μM) for the indicated times. HA-tagged receptors were immunoprecipitated with anti-HA magnetic beads and the IP was probed with an anti-GFP antibody that recognizes YFP (MBL, International) and subsequently blots were stripped and reprobed with an antibody that detects HA (top panel). βarrestin2 and βarrestin2-SUMO1 bands were divided by respective HA-D2R band and the ratios are plotted for each sample from three independent experiments (bottom panel). Statistical comparison was done using two-way ANOVA and shown as means±SEM of 3 independent experiments. * p<0.05 versus all other conditions, two-way ANOVA, Holm-Sidak's comparison.

FIG. 6 shows BRET analysis of protein-protein interactions between agonist-activated D2R and βarrestin2, βarrestin2-SUMO1, and βarrestin2-Ub as a measure of recruitment to the D2R, according to certain aspects of this disclosure. HEK-293 cells were transiently transfected with D2R-RlucII along with YFP-βarr2-WT, YFP-βarr2-SUMO1 or YFP-βarr2-Ub and incubated with the indicated concentrations of dopamine for 15 min. BRET signals were measured as described in Example 1 herein, and changes following agonist treatment are expressed as a percentage of the BRET signal observed in unstimulated cells (top panel). Dose-response curves generated were compared by two-way ANOVA, which revealed an effect of drug (P<0.0001) and concentration (P<0.0001) as well as an interaction (P<0.0001). The histogram (bottom panel) represents Bmax values of agonist induced BRET as means±SEM of 4 independent experiments performed in duplicates. Significance of changes in energy transfer was established by one-way ANOVA comparing signals measured for the recruitment of βarr2 fusion proteins to D2R-RlucII *p<0.01; **p<0.001.

FIG. 7 shows that βarrestin2 and βarrestin2-SUMO1 bind to agonist activated V₂R with similar affinities, according to certain aspects of this disclosure. Shown are BRET measurements, which were obtained as described in Example 1 herein and correspond to the vasopressin-induced (15 min stimulation) βarrestin2 recruitment to the V₂R. Changes following agonist treatment are expressed as a percentage of the BRET signal observed in unstimulated cells (top panel). The histogram (bottom panel) represents Bmax values of agonist-induced BRET as means±SEM of 3 independent experiments performed in duplicates. Lack of significance (“ns”) of changes in energy transfer was evaluated using unpaired t-test.

FIG. 8 shows the protein-protein association between HA-V₂R with and without arginine-vasopressin (AVP) agonist treatment and either YFP-βarrestin2 or YFP-βarrestin2-SUMO1, according to certain aspects of this disclosure. The protein interactions were determined as in FIG. 5, and the blot panels shown are from one of two independent experiments.

FIG. 9 shows protein levels in cells expressing RanGAP1 and YFP-βarrestin2, according to certain aspects of this disclosure. HEK-293T cells stably transfected with β₂AR were transfected with vector or YFP-βarrestin2. 48 hours later, cells were solubilized and analyzed for RanGAP1, βarrestin2 and GAPDH by Western blotting (top panel). The unmodified RanGAP1 band (70 kDa) was divided by cognate GAPDH and plotted as a ratio. The bar graph (bottom panel) summarizes values from three independent experiments. *p=0.006, unpaired t test.

FIG. 10 shows the effect of βarrestin2 depletion on RanGAP1 levels, according to certain aspects of this disclosure. HEK-293T cells stably expressing β₂AR were transfected with either control siRNA or siRNA targeting βarr2. 48 h later, transfected cells were serum-starved for 1 h and then stimulated with 1 μM isoproterenol for 20 min. Cell lysates were resolved on 10% SDS-gels and immunoblotted for the indicated proteins (top panel). The ratios of the band intensity of RanGAP1 (˜70 kDa) and GAPDH were plotted as means±SEM from three independent experiments (bottom panel). *, p<0.05 versus control, two-way ANOVA and Holm-Sidak's test.

FIG. 11 shows the effect of βarrestin2 SUMOylation on RanGAP1 levels and RanGAP1 SUMOylation, according to certain aspects of this disclosure. HEK-293T cells were transiently transfected with indicated plasmids and subjected to coimmunoprecipitation with GFP IgG (3E6, Thermo Fisher) to immunoprecipitate YFP-βarrestin2 or YFP-βarrestin2-SUMO1 (“IP: YFP”). IPs were probed for endogenous RanGAP1, and for βarrestins. Cell lysates were also blotted to determine total cell protein levels (“Lysates”). Blots shown are from one of three independent experiments.

FIG. 12 shows the effect of βarrestin2 depletion on RanGAP1 levels, according to certain aspects of this disclosure, similarly to FIG. 10, but in HEK-293 cells rather than HEK-293T cells. HEK-293 cells stably expressing β₂AR were transfected with either control siRNA or siRNA targeting βarr2. 48 h later, transfected cells were serum-starved for 1 h and then stimulated with 1 μM isoproterenol for 20 min. Cell lysates were resolved on 10% SDS-gels and immunoblotted for the indicated proteins (top panel). The ratios of the band intensity of RanGAP1 (˜70 kDa) and GAPDH were plotted as means±S.E.M from three independent experiments (bottom panel). *, p<0.05 versus control, two-way ANOVA and Holm-Sidak's test.

FIG. 13 shows the effect of βarrestin2 SUMOylation on RanGAP1 levels and RanGAP1 SUMOylation, according to certain aspects of this disclosure, similarly to FIG. 11, but in HEK-293 cells rather than HEK-293T cells. HEK-293T cells were transiently transfected with indicated plasmids and subjected to coimmunoprecipitation with Flag affinity gel, and the bound endogenous RanGAP1 was detected with RanGAP1 antibody (Ab92360, Abcam). The blots were stripped and reprobed for βarrestins. The blot panels are from one of three independent experiments.

FIG. 14 shows that a βarrestin1-SUMO1 fusion protein displays stronger association with agonist-activated β₂AR than wild type βarrestin1, according to certain aspects of this disclosure. The bar graph summarizes the extent of colocalization of βarrestin1 and βarrestin1-SUMO1 with the β₂AR observed in confocal images of HEK-293 cells expressing mYFP-βarrestin1 or mYFP-βarrestin1-SUMO1 and β₂AR, with or without 5 min of isoproterenol stimulation.

FIG. 15 shows that a βarrestin1-SUMO1 fusion protein associates robustly with agonist-activated β₂AR, according to certain aspects of this disclosure. The bar graph summarizes the BRET ratio corresponding to the recruitment of βarrestin1 or βarrestin1-SUMO1 fusion protein to the β₂AR with or without isoproterenol (β₂AR agonist) stimulation, as described in Example 1 herein. While βarrestin1 showed a weak association, βarrestin1-SUMO1 showed significantly more binding to the β₂AR. * p<0.01 compared with all other samples, one-way ANOVA, Bonferroni post hoc test.

FIG. 16 shows that a βarrestin1-SUMO1 fusion protein associates robustly with agonist-activated glucagon receptor (GCGR), according to certain aspects of this disclosure. The bar graph summarizes the BRET ratio corresponding to the recruitment of βarrestin1 or βarrestin1-SUMO1 fusion protein to the GCGR with or without glucagon (GCGR agonist) stimulation, as described in Example 1 herein. While βarrestin1 showed a weak association, βarrestin1-SUMO1 showed significantly more binding to the GCGR. * p<0.01, **p<0.001, compared to βarrestin1, two one-way ANOVA, Bonferroni post hoc test.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

### I. Introduction

In the presence of continuous agonist stimulation, GPCRs are phosphorylated by specific GPCR kinases (GRKs), and the recruitment of arrestins to the phosphorylated GPCRs eventually terminates G protein signaling and leads to a coordinated process of receptor desensitization, inactivation, and internalization. Arrestins also facilitate the formation of multi-molecular complexes and provide a means for G protein-independent signaling of GPCRs, including those involving mitogen-activated protein (MAP) kinases, receptor and non-receptor tyrosine kinases, phosphatidylinositol 3-kinases (PI3K) and others.

Arrestins are a small family of proteins which include arrestin-1 (also known as visual arrestin or rod arrestin), beta-arrestin-1 (βarrestin1; also known as arrestin-2), beta-arrestin-2 (βarrestin2; also known as arrestin-3), and arrestin-4 (also known as X-arrestin or cone arrestin). In mammals, arrestin-1 and arrestin-4 are largely confined to photoreceptors, whereas βarrestin1 and βarrestin2 are ubiquitous.

βarrestin1 and βarrestin2 (“beta-arrestins” or “βarrestins”) are highly conserved proteins that display high affinity interaction with agonist-activated GPCRs that are phosphorylated on specific serine/threonine residues by GPCR kinases (GRKs). See DeWire et al. 2007. Annu Rev Physiol 69:483-510. βarrestins and GRKs uncouple the agonist-activated GPCRs from cognate heterotrimeric G proteins, thereby downregulating or inactivating G protein-dependent signaling. βarrestins in turn provoke GPCR endocytosis and additionally scaffold kinases resulting in βarrestin-dependent signal transduction. See DeWire et al., supra and Luttrell et al. 2018. Sci Signal 11. In addition to GPCRs, βarrestins can bind other types of cell-surface receptors, ion channels, and engage many signaling and biochemical pathways. See Shenoy and Lefkowitz. 2011. Trends Pharmacol. Sci. 32:521-533.

Arrestin proteins are regulated not only by GPCR recruitment, but also by ubiquitination. See Gurevich and Gurevich. 2015. Prog Mol Biol Transl Sci 132:1-14. For example, GPCR activation triggers ubiquitination of lysine residues in βarrestin2 and the sites of ubiquitination as well as the kinetics and patterns of ubiquitination have distinct correlation to particular GPCR:βarrestin complexes. See Shenoy et al. 2007. J Biol Chem 282:29549-29562 and Jean-Charles et al. 2016. Prog Mol Biol Transl Sci 141:339-369. Ubiquitinated βarrestin2 possesses greater binding affinity than non-ubiquitinated βarrestin2 with (i) activated GPCRs, (ii) clathrin subunits and (iii) components of ERK signaling (c-Raf and ERK), which suggests a tight relationship between βarrestin ubiquitination status, endocytosis, and the transmission of βarrestin-dependent signaling. See Shenoy et al. 2007, supra.

Arrestin proteins are also regulated by covalent modification by ubiquitin and SUMO (small ubiquitin like modifier) or SUMOylation. See, e.g., Kommaddi and Shenoy. 2013. Prog Mol Biol Trans' Sci 118:175-204, Wyatt et al. 2011. J Biol Chem 286:3884-3893, and Xiao et al. 2015. J Biol Chem 290:1927-1935. SUMO and ubiquitin are ubiquitin-like proteins (UBLs), a family of small proteins involved in post-translational modification of other proteins in a cell, usually with a regulatory function. See, e.g., Hochstrasser. 2009. Nature 458:422-429. UBLs that are capable of conjugation (sometimes known as Type I) have a characteristic sequence motif consisting of one to two glycine residues at the C-terminus, through which covalent conjugation occurs. Typically, UBLs are expressed as inactive precursors and must be activated by proteolysis of the C-terminus to expose the active glycine. Almost all such UBLs are ultimately linked to another protein. UBLs that do not exhibit covalent conjugation (Type II) often occur as protein domains genetically fused to other domains in a single larger polypeptide chain, and may be proteolytically processed to release the UBL domain or may function as protein-protein interaction domains. As used in the context of this disclosure, the term “ubiquitin-like protein” or “UBL” refers only to Type I UBLs. Type II UBLs are outside the context of this disclosure.

Ubiquitin and SUMO share little sequence identity but adopt similar structural conformations, and both require a three step enzyme cascade for substrate modification. See Saitoh et al. 1997. Trends Biochem Sci 22:374-376. SUMOylation is generally targeted to a canonical protein sequence (Ψ-K-X-D/E), where Ψ is an aliphatic amino acid, K is the target site for the covalent modification by SUMO, X is any amino acid and is followed by an acidic residue. The canonical SUMOylation site, along with the 4 residues flanking the site on either side, is fully conserved in rat, mouse, human, and bovine βarrestin2, in the sequence LDGQLKHEDTNL (SEQ ID NO:14; canonical SUMOylation site underlined and target site for covalent modification by SUMO shown in bold).

SUMOylation and ubiquitination are dynamic and reversed by cognate de-SUMOylases and de-ubiquitinases, respectively. Many UBLs may regulate arrestin protein function. Because these modifications are dynamic and potentially short-lived, deducing their impact on arrestin is difficult. Provided herein are fusion proteins in which a UBL is fused to an arrestin polypeptide. In some embodiments, the fusion proteins are resistant to enzymatic activity to remove the UBL (e.g., de-SUMOylation). In some embodiments, the fusion proteins behave similarly to an endogenous arrestin protein modified with ubiquitin or a UBL. Also provided are methods of using the fusion proteins to assess arrestin trafficking, localization, and other functions including arrestin-mediated GPCR signaling.

### II. Polynucleotides and Polypeptides

Provided in this disclosure are fusion proteins in which a ubiquitin-like protein (UBL) is fused to an arrestin polypeptide. In some embodiments, the fusion proteins are resistant to de-SUMOylation. In some embodiments, the fusion proteins provided herein display increased binding to one or more G protein-coupled receptors (GPCRs) and/or altered subcellular localization upon expression in cells (e.g., measured relative to the wild-type form of the arrestin polypeptide unmodified with a UBL). In some embodiments, as described herein, the fusion proteins are able to bind to one or more GPCRs, including, but not limited to, angiotensin type 1a receptor (AT₁ₐR), β₂ adrenergic receptor (β₂AR), D₂ dopamine receptor (D2R), β₁ adrenergic receptor (β₁AR), D₁ dopamine receptor (D1R), V₂ vasopressin receptor (V₂R) and glucagon receptor (GCGR). In some embodiments, as shown in the Examples herein, the fusion proteins display increased binding to one or more GPCRs including, but not limited to, β₂ adrenergic receptor (β₂AR), D₂ dopamine receptor (D2R), β₁ adrenergic receptor (β₁AR), D₁ dopamine receptor (D1R), and glucagon receptor (GCGR).

As used throughout, “increased binding” of one protein to another protein may be measured in a variety of ways. For example, increased binding may be measured as a longer duration of interaction between two proteins, an increased frequency of interactions between two proteins (i.e., a higher proportion of the available proteins are interacting with each other), a stronger binding strength (affinity), and/or a more rapid initiation of interaction upon stimulation of one or both of the proteins (e.g., with a GPCR agonist compound, as described herein). Increased binding may also be measured via measurement of a known downstream effect of said binding. For example, increased binding of an arrestin protein to a GPCR may lead to decreased G protein-dependent signaling and/or prolonged desensitization of the GPCR. As such, any suitable assay for detecting these downstream effects may be used to measure increased binding of an arrestin protein to a GPCR. Multiple methods of detecting increased binding are described herein.

Provided herein is a fusion protein comprising an arrestin polypeptide fused to a ubiquitin-like protein (UBL). In some embodiments, the fusion protein comprises an amino acid sequence having at least 80% identity, for example, at least about 80%, 81%, 82%, 83%, 84%, 85%, 86%, 87%, 88%, 89%, 90%, 91%, 92%, 93%, 94%, 95%, 96%, 97%, 98%, or 99%, to SEQ ID NO:1 (rat βarrestin2 polypeptide fused to human SUMO1) or SEQ ID NO:2 (rat βarrestin1 polypeptide fused to human SUMO1).

As used throughout, a “fusion protein” is a protein comprising two different polypeptide sequences, i.e. an arrestin polypeptide sequence and a UBL polypeptide sequence, that are joined or linked to form a single polypeptide. In some embodiments, the two amino acid sequences are encoded by separate nucleic acid sequences that have been joined so that they are transcribed and translated to produce a single polypeptide. In some embodiments, the fusion protein comprises, in the following order, an arrestin polypeptide and a UBL polypeptide.

An arrestin polypeptide of the present disclosure comprises the amino acid sequence of all or part of a protein belonging to the arrestin family of proteins. In some embodiments, the arrestin protein or portion thereof retains the function of the full length protein. In some embodiments, the arrestin polypeptide of the fusion proteins provided herein comprises at least 80%, for example, at least 82%, 84%, 88%, 90%, 91%, 92%, 93%, 94%, 95%, 96%, 97%, 98%, or 99% of the amino acid sequence of an arrestin protein (e.g., βarrestin2, βarrestin1, arrestin-1, or arrestin-2). In some instances, the arrestin protein is a mammalian arrestin protein (e.g., from human, non-human primate, mouse, rat, rabbit, pig, goat, sheep, horse, or cow). For example, the arrestin protein can be a human arrestin protein (e.g., human βarrestin1 or human βarrestin2). In another example, the arrestin protein can be a rat arrestin protein (e.g., rat βarrestin1 or rat βarrestin2). In some instances, the arrestin protein is a non-mammalian arrestin protein (e.g., from Drosophila species, Danio species, or from other organisms of interest). In some embodiments, the arrestin polypeptide comprises an amino acid sequence having at least 80% identity, for example, at least about 80%, 81%, 82%, 83%, 84%, 85%, 86%, 87%, 88%, 89%, 90%, 91%, 92%, 93%, 94%, 95%, 96%, 97%, 98%, or 99%, to the amino acid sequence of rat βarrestin2 (SEQ ID NO:3), rat βarrestin1 (SEQ ID NO:4), human βarrestin2 (SEQ ID NO:5), or human βarrestin1 (SEQ ID NO:6).

A UBL of the present disclosure comprises all or part of a ubiquitin protein or a protein belonging to the ubiquitin-like protein family. In some embodiments, the UBL retains the function of the full length protein. In some embodiments, the UBL of the fusion proteins provided herein comprises at least 80% (e.g., at least 82%, at least 84%, at least 88%, at least 90%, at least 92%, at least 94%, at least 96%, at least 98%, at least 99%, or 100%) of the amino acid sequence of a UBL. In some instances, the UBL is mammalian UBL (e.g., human, non-human primate, mouse, rat, rabbit, pig, goat, sheep, horse, or cow). For example, the UBL can be a human UBL. In some instances, the UBL is a non-mammalian UBL (e.g., from Drosophila species, Danio species, or from other species of interest). In some embodiments, the UBL comprises an amino acid sequence having at least 80% identity, for example, at least about 80%, 81%, 82%, 83%, 84%, 85%, 86%, 87%, 88%, 89%, 90%, 91%, 92%, 93%, 94%, 95%, 96%, 97%, 98%, or 99%, to the amino acid sequence of human SUMO1 (SEQ ID NO:7), human SUMO2 (SEQ ID NO:8), human SUMO3 (SEQ ID NO:9), human ubiquitin (SEQ ID NO:10), human ISG15 (SEQ ID NO:11), human NEDD8 (SEQ ID NO:12), or human FAT10 (SEQ ID NO:13).

In some embodiments, the arrestin protein is fused to the UBL via a peptide linker. The peptide linker can be from about 2 to about 100 amino acids in length. For example, the linker can be a linker of from about 2 to about 5 amino acids in length, from about 2 to about 10 amino acids in length, from about 2 to about 20 amino acids in length, from about 2 to about 25 amino acids in length, from about 2 to about 30 amino acids in length, from about 2 to about 35 amino acids in length, from about 2 to about 40 amino acids in length, from about 2 to about 45 amino acids in length, from about 2 to about 50 amino acids in length, from about 2 to about 55 amino acids in length, from about 2 to about 60 amino acids in length, from about 2 to about 65 amino acids in length, from about 2 to about 70 amino acids in length, from about 2 to about 75 amino acids in length, from about 2 to about 80 amino acids in length, from about 2 to about 85 amino acids in length, from about 2 to about 90 amino acids in length, from about 2 to about 95 amino acids in length, from about 2 to about 100 amino acids in length.

In some embodiments, the peptide linker can be from about 1% to about 10%, for example, about 2% to about 5%, about 1% to about 4%, about 1% to about 6%, about 1% to about 8%, about 3% to about 6%, about 3% to about 8%, about 4% to about 7%, about 4% to about 10%, or about 5% to about 10%) of the total length of the fusion protein. In some embodiments, the linker sequence may be optimized to produce desired effects in the fusion protein. In some embodiments, a majority of the amino acid residues of the linker sequence can comprise alanine and/or glycine residues. In some embodiments, the linker sequence may include one or more acidic residues.

Exemplary peptide linkers include, but are not limited to, peptide linkers comprising any of SEQ ID NO: 15 (SGSETPGTSESATPE), SEQ ID NO: 16 (SGSETPGTSESATPES), SEQ ID NO: 17 ((GGGGS)₃), SEQ ID NO: 18 ((GGGGS)₁₀), SEQ ID NO: 19 ((GGGGS)₂₀), SEQ ID NO: 20 (A(EAAAK)₃A), or SEQ ID NO: 21 (A(EAAAK)₁₀A).

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds. Variants of the polypeptides of this disclosure retain their respective biological activity. For example, variants of the arrestin polypeptide retain the biological function of the full length, native sequence arrestin polypeptide. In another example, variants of the UBL polypeptide retain the biological function of the full length, native sequence UBL.

Modifications to any of the polypeptides or proteins provided herein are made by known methods. By way of example, modifications are made by site specific mutagenesis of nucleotides in a nucleic acid encoding the polypeptide, thereby producing a DNA encoding the modification, and thereafter expressing the DNA in recombinant cell culture to produce the encoded polypeptide. Techniques for making substitution mutations at predetermined sites in DNA having a known sequence are well known. For example, M13 primer mutagenesis and PCR-based mutagenesis methods can be used to make one or more substitution mutations. Any of the nucleic acid sequences provided herein can be codon-optimized to alter, for example, maximize expression, in a host cell or organism.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-587 (2013); Xie et al. “Adding amino acids to the genetic repertoire,” 9(6): 548-54 (2005)); and all references cited therein. B and γ amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

A biologically active variant of an arrestin polypeptide in the context of this disclosure may differ by as few as about 1-15 amino acid residues, as few as about 1-10, such as about 6-10, as few as 5, as few as 4, as few as 3, as few as 2, or as few as 1 amino acid residue. In specific embodiments, the arrestin polypeptide can comprise an N-terminal or a C-terminal truncation, which can comprise at least a deletion of 10, 15, 20, 25, 30, 35, 40, 45, 50 amino acids or more from either the N-terminal or C-terminal end of the polypeptide.

Any of the polypeptides and fusion proteins described herein can further comprise a detectable moiety, for example, a fluorescent protein or fragment thereof. In some embodiments, the fusion protein may comprise a BRET fluorescence donor or a BRET fluorescence acceptor as described in Section IV. In some embodiments, the fusion proteins provided herein comprise a detectable moiety or a BRET fluorescence donor or acceptor at the N-terminal end, at the C-terminal end, and/or internally (e.g., between the arrestin polypeptide and the UBL). Examples of fluorescent proteins include, but are not limited to, yellow fluorescent protein (YFP, for example, Venus), green fluorescent protein (GFP), and red fluorescent protein (RFP) as well as derivatives, for example, mutant derivatives, of these proteins. See, for example, Chudakov et al. “Fluorescent Proteins and Their Applications in Imaging Living Cells and Tissues,” Physiological Reviews 90(3): 1103-1163 (2010); and Specht et al., “A Critical and Comparative Review of Fluorescent Tools for Live-Cell Imaging,” Annual Review of Physiology 79: 93-117 (2017)). Additional discussion of suitable BRET fluorescence donors and acceptors is provided in Section IV.

Any of the polypeptides described herein can further comprise an affinity tag, for example a polyhistidine tag (e.g., (His)₆ (SEQ ID NO:22)), an HA tag (e.g., YPYDVPDYA (SEQ ID NO:23)), albumin-binding protein, alkaline phosphatase, an AU1 epitope, an AU5 epitope, a biotin-carboxy carrier protein (BCCP), a FLAG epitope (e.g., DYKDDDDK (SEQ ID NO:24), or a MYC epitope (e.g., EQKLISEEDL (SEQ ID NO:25)), to name a few. See, Kimple et al. “Overview of Affinity Tags for Protein Purification, Curr. Protoc. Protein Sci. 73: Unit-9.9 (2013).

Recombinant nucleic acids encoding any of the polypeptides described herein are also provided. For example, a recombinant nucleic acid encoding a polypeptide that has at least 90%, for example, at least about 91%, 92%, 93%, 94%, 95%, 96%, 97%, 98% or 99%, identity to any one of SEQ ID NOs 1-25 is also provided.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues. See Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994).

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix. See Henikoff & Henikoff, Proc. Natl. Acad. Sci. USA 89:10915 (1989).

The BLAST algorithm also performs a statistical analysis of the similarity between two sequences. See, e.g., Karlin & Altschul, Proc. Nat'l. Acad. Sci. USA 90:5873-5787 (1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

### III. Constructs, Vectors, and Host Cells

Also provided is a DNA construct comprising a promoter operably linked to a recombinant nucleic acid described herein. A nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription. The promoter can be a eukaryotic or a prokaryotic promoter. In some embodiments the promoter is an inducible promoter. In some embodiments, the promoter is a constitutive promoter. In some embodiments, the promoter is derived from an endogenous promoter that drives expression of an arrestin family protein or a UBL in a cell or in vitro expression system. In some embodiments, the promoter is derived from human cytomegalovirus (CMV), e.g., the human CMV immediate early enhancer-containing promoter.

The recombinant nucleic acids provided herein can be included in expression cassettes for expression in a host cell or an organism of interest. The cassette will include 5′ and 3′ regulatory sequences operably linked to a recombinant nucleic acid provided herein that allows for expression of the modified polypeptide. The cassette may additionally contain at least one additional gene or genetic element to be cotransformed into the cell or organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette may additionally contain a selectable marker gene. The expression cassette will include in the 5′ to 3′ direction of transcription: a transcriptional and translational initiation region (i.e., a promoter), a polynucleotide of the invention, and a transcriptional and translational termination region (i.e., termination region) functional in the cell or organism of interest. The promoters of the invention are capable of directing or driving expression of a coding sequence in a host cell. The regulatory regions (i.e., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.); Davis et al., eds. (1980) Advanced Bacterial Genetics (Cold Spring Harbor Laboratory Press), Cold Spring Harbor, N.Y., and the references cited therein.

The expression cassette can also comprise a selectable marker gene for the selection of transformed cells. Marker genes include genes conferring antibiotic resistance, such as those conferring hygromycin resistance, ampicillin resistance, gentamicin resistance, neomycin resistance, to name a few. Additional selectable markers are known and any can be used.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be used.

Further provided is a vector comprising a nucleic acid or expression cassette set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. See generally, Sambrook et al. Molecular Cloning: A Laboratory Manual, 4^{th} ed., Cold Spring Harbor Laboratory Press, Cold Spring Harbor, 2012. The vector, for example, can be a plasmid.

There are numerous E. coli expression vectors known to one of ordinary skill in the art, which are useful for the expression of a nucleic acid. Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used. Provided herein is a nucleic acid encoding a polypeptide of the present invention, wherein the nucleic acid can be expressed by a yeast cell. More specifically, the nucleic acid can be expressed by Pichia pastoris or S. cerevisiae.

Mammalian cells also permit the expression of proteins in an environment that favors important post-translational modifications such as folding and cysteine pairing, addition of complex carbohydrate structures, and secretion of active protein. Vectors useful for the expression of active proteins in mammalian cells are known in the art and can contain genes conferring hygromycin resistance, geneticin or G418 resistance, or other genes or phenotypes suitable for use as selectable markers, or methotrexate resistance for gene amplification. A number of suitable host cell lines capable of secreting intact human proteins have been developed in the art, and include CHO cells, HeLa cells, HEK-293 cells, HEK-293T cells, U2OS cells, or any other primary or transformed cell line. Other suitable host cell lines include COS-7 cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc.

The expression vectors described herein can also include the nucleic acids as described herein under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

Insect cells also permit the expression of the polypeptides. Recombinant proteins produced in insect cells with baculovirus vectors undergo post-translational modifications similar to that of wild-type mammalian proteins.

Also provided herein are host cells comprising the recombinant nucleic acids, DNA constructs, and/or vectors described herein as well as methods of making such cells

A host cell comprising a nucleic acid or a vector described herein is provided. The host cell can be an in vitro, ex vivo, or in vivo host cell. Host cells as provided herein are capable of expressing the fusion protein. Cell populations of any of the host cells described herein are also provided. In some embodiments, the cell population comprises a plurality of cells, wherein the plurality of cells comprise a recombinant nucleic acid encoding the fusion protein as described herein. In some embodiments, the cell population comprises a plurality of cells, wherein the plurality of cells comprises a DNA construct encoding the fusion protein as described herein. In some embodiments, the cell population comprises a plurality of cells, wherein the plurality of cells comprises a vector comprising a recombinant nucleic acid or a DNA construct encoding the fusion protein as described herein. In some embodiments, the cell population comprises a plurality of cells, wherein the plurality of cells comprise a plurality of any of the host cells described herein. In some embodiments, a plurality of cells of any of the cell populations described herein express a fusion protein as described herein.

In some embodiments, the provided cells express the fusion protein stably or transiently. Stable expression of the fusion protein in a cell refers to integration of any of the nucleic acids, DNA constructs, or vectors described herein into the genome of the cell, thereby allowing the cell to express the fusion protein. Transient expression refers to expression of the fusion protein directly from any of the nucleic acids, DNA constructs, and/or vectors following introduction into the cell (i.e., the gene encoding the fusion protein is not integrated into the genome of the cell).

In some embodiments, the provided cells express the fusion protein constitutively or inducibly. Constitutive expression refers to ongoing, continuous expression of a gene (i.e., of a protein), whereas inducible expression refers to gene (protein) expression that is responsive to a stimulus. Inducible expression is generally regulated via an inducible promoter, a description of which is included above.

A cell culture comprising one or more host cells described herein is also provided. Methods for the culture and production of many cells, including cells of bacterial (for example E. coli and other bacterial strains), animal (especially mammalian), and archebacterial origin are available in the art. See e.g., Sambrook, supra; Ausubel, ed. (1995) Current Protocols in Molecular Biology, John Wiley & Sons, as well as Freshney (1994) Culture of Animal Cells, a Manual of Basic Technique, 3^{rd} Ed., Wiley-Liss, New York and the references cited therein; Doyle and Griffiths (1997) Mammalian Cell Culture: Essential Techniques John Wiley and Sons, NY; Humason (1979) Animal Tissue Techniques, 4^{th} Ed. W.H. Freeman and Company; and Ricciardelli, et al., (1989) In vitro Cell Dev. Biol. 25:1016-1024.

The host cell can be a prokaryotic cell, including, for example, a bacterial cell. Alternatively, the cell can be a eukaryotic cell, for example, a mammalian cell. In some embodiments, the cell can be a HEK-293T cell, a HEK-293 cell, a Chinese hamster ovary (CHO) cell, a U2OS cell, or any other primary or transformed cell. In some embodiments, the cell can be a COS-7 cell, a HELA cell, an avian cell, a myeloma cell, a Pichia cell, an insect cell or a plant cell. A number of other suitable host cell lines have been developed and include myeloma cell lines, fibroblast cell lines, and a variety of tumor cell lines such as melanoma cell lines. The vectors containing the nucleic acid segments of interest can be transferred or introduced into the host cell by well-known methods, which vary depending on the type of cellular host.

As used herein, the phrase “introducing” in the context of introducing a nucleic acid into a cell refers to the translocation of the nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease, a transcription activator-like effector nuclease (TALEN), a zinc finger nuclease (ZFN), or a megaTAL (MT) can also be used to introduce a nucleic acid, for example, a nucleic acid encoding a fusion protein described herein, into a host cell. See Li et al. Signal Transduction and Targeted Therapy 5, Article No. 1 (2020).

The CRISPR/Cas9 system, an RNA-guided nuclease system that employs a Cas9 endonuclease, can be used to edit the genome of a host cell or organism. The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Any of the fusion proteins described herein can be purified or isolated from a host cell or population of host cells. For example, a recombinant nucleic acid encoding any of the fusion proteins described herein can be introduced into a host cell under conditions that allow expression of the fusion protein. In some embodiments, the recombinant nucleic acid is codon-optimized for expression. After expression in the host cell, the fusion protein can be isolated or purified using purification methods known in the art. As used herein, the term “isolated” or “purified” means that the protein is substantially free of other components found in the cell.

### IV. Methods

In another aspect, provided herein are methods for detecting subcellular localization patterns of one or more arrestin proteins and/or one or more G protein-coupled receptors (GPCRs), detecting protein-protein interactions of one or more arrestin proteins with one or more GPCRs, and identifying whether a drug compound impacts arrestin-mediated GPCR signaling. The methods according to the present disclosure provide substantial improvement in the ability to study various aspects related to arrestin-mediated GPCR signaling, including, but not limited to, arrestin protein regulation, arrestin protein localization, binding interactions between arrestin proteins and GPCRs, arrestin-induced GPCR internalization, and downstream signaling effects induced by arrestin-mediated GPCR signaling. The various methods provided herein may comprise detecting protein subcellular localization and/or protein-protein interactions using a variety of known methods. In some instances, the methods provided herein are useful for the screening of drug compounds that impact arrestin-mediated GPCR signaling.

In some instances, the methods provided herein are also useful for detecting subcellular localization patterns, protein-protein interactions, and other functional characteristics of non-GPCR proteins with which arrestin proteins interact. In some embodiments, the methods may be useful for assessing whether a drug compound impacts arrestin-mediated signaling that is independent of arrestin-GPCR interaction. In addition to GPCRs, arrestin proteins are known to bind to proteins from other categories, including, but not limited to, non-GPCR cell-surface receptors (e.g., single transmembrane receptors), ion channels, endocytic proteins, and nuclear membrane proteins (e.g., RanGAP1). Arrestin proteins are also known to engage many signaling and biochemical pathways. See Shenoy and Lefkowitz. 2011, supra. In some embodiments, the methods provided herein are useful for assessing functional characteristics (e.g., subcellular localization patterns, protein-protein interactions, etc.) of non-GPCR signaling proteins. As used throughout, “signaling protein” refers to any protein involved in any aspect of cell signaling. As such, a signaling protein may be a GPCR, a cell-surface receptor, an ion channel, an endocytic protein, a nuclear membrane protein, a cytokine, a hormone, or any other type of protein that can be involved in cell signaling.

In some embodiments, the methods herein are useful for assessing functional characteristics of a single transmembrane receptor protein with which an arrestin protein interacts. Such single transmembrane receptor proteins include, but are not limited to, insulin-like growth factor 1 receptor (IGF1-R), a transforming growth factor beta receptor (TGF-R), a Notch receptor, a receptor tyrosine kinase (e.g., an insulin receptor), an interleukin receptor, or a toll-like receptor.

In some embodiments, the methods herein are useful for assessing functional characteristics of a non-receptor protein with which an arrestin protein interacts. In some embodiments, the non-receptor protein is an endocytic protein. In some embodiments, the endocytic protein is a ras-related nuclear protein (Ran) or a member of the Rab protein family. In some embodiments, the non-receptor protein is a protein that localizes to the nuclear membrane. In some embodiments, the non-receptor protein is RanGAP1. In some embodiments, the non-receptor protein is a mitogen-activated protein kinase (e.g., an extracellular signal-regulated kinase (ERK), a p38 mitogen-activated protein kinase, or a c-Jun N-terminal kinase (JNK)). In some embodiments, the non-receptor protein is tumor protein P53 (p53). In some embodiments, the non-receptor protein is mouse double minute 2 homolog (MDM2).

For ease and clarity of discussion, the methods provided herein are described with reference to GPCRs. However, one of skill in the art will recognize that the fusion proteins and methods of use thereof provided herein may also be useful for assessing any other arrestin-mediated signaling pathway or a functional characteristic of any protein that interacts with an arrestin protein. Unless specifically noted, any method described herein that refers to GPCRs may be modified to encompass any non-GPCR arrestin interaction partner, known or unknown.

Provided herein is a method for detecting a protein subcellular localization pattern comprising: (a) providing a plurality of cells that express a fusion protein described herein; and (b) detecting the subcellular localization pattern of the fusion protein in the plurality of cells. In some embodiments, the plurality of cells also express one or more GPCRs to which the fusion protein binds. In such instances, the method may further comprise detecting the subcellular localization pattern of the one or more GPCRs, i.e., detection of the subcellular localization pattern of the fusion protein may also provide detection of the subcellular localization pattern of the one or more GPCRs to which the fusion protein is bound.

Also provided herein is a method for detecting protein-protein interaction of an arrestin protein and one or more GPCRs comprising: (a) providing a plurality of cells that express a fusion protein described herein and the GPCR(s); (b) detecting the protein-protein interaction of the fusion protein with the GPCR(s) in the plurality of cells.

In the methods provided herein, detecting the subcellular localization pattern of a fusion protein or the interaction of a fusion protein with a GPCR may be performed using any suitable method. In some embodiments, detecting the subcellular localization pattern of a fusion protein or the interaction of a fusion protein with a GPCR is performed by immunostaining, confocal microscopy, bioluminescence energy transfer (BRET), affinity chromatography, and/or immunoprecipitation. In some embodiments, detecting the subcellular localization pattern of a fusion protein comprising a detectable marker (e.g., YFP) is performed by detecting the detectable marker (e.g., by confocal microscopy). In some embodiments, detecting the subcellular localization pattern of a fusion protein may comprise microscopic analysis of fixed or live cells expressing the fusion protein (e.g., through immunostaining), cellular fractionation followed by protein analysis (e.g., by mass spectrometry), or any other assay for detecting subcellular localization known in the art. In some embodiments, the subcellular localization pattern of a GPCR may be detected by detecting the subcellular localization pattern of a fusion protein described herein that is bound to the GPCR.

Detecting the interaction of a fusion protein with a GPCR may be performed using any known assay for detecting protein-protein interaction. See, e.g., Rao et al. 2014. International Journal of Proteomics vol. 2014, art. ID 147648. In some embodiments, a fusion protein is immunoprecipitated, followed by analysis of coimmunoprecipitated proteins for a GPCR of interest. In some embodiments, a GPCR of interest is immunoprecipitated, followed by analysis of coimmunoprecipitated proteins for a fusion protein described herein. In some embodiments, microscopic analysis of fixed or live cells using any of the techniques described herein (e.g., immunostaining or detection of fluorophore-tagged proteins) may show colocalization of two proteins, which may suggest protein-protein interaction.

In some embodiments, detecting the interaction of a fusion protein with a GPCR may be performed using BRET. See, e.g., Kobayashi et al. 2019. Nature Protocols 14:1084-1107. BRET is a transfer of energy between a luminescence donor and a fluorescence acceptor. Because BRET occurs when the distance between the donor and acceptor is less than 10 nm, and because BRET efficiency is dependent on the inverse sixth power of the intermolecular separation, it is useful as a proximity-based assay to monitor protein-protein interactions in live cells. For ease of reference, the terminology used in this disclosure makes reference primarily to luminescence molecules that can act as a luminescence donor and fluorophore molecules that can act as a fluorescence acceptor for use of BRET in the provided methods. This should not be interpreted as excluding other types of donor and acceptor molecules (e.g., non-fluorophore molecules).

In the methods provided herein, BRET luminescence donors may include any suitable molecule capable of luminescence, with or without addition of a substrate. Luciferase enzymes are generally well-suited for use as luminescence donors. Useful luciferase enzymes may be those isolated from species including, but not limited to, Photinus pyralis, Luciola cruciate, Luciola italic, Luciola lateralis, Luciola mingrelica, Photuris pennsylvanica, Pyrophorus plagiophthalamus, Phrixothrix hirtus, Renilla reniformis, Gaussia princeps, Cypridina noctiluca, Cypridina hilgendorfii, Metridia longa, Oplophorus gracilorostris. Luciferase enzymes may be useful in their native state, or they may be mutated or engineered to improve properties such as stability and luminescence. Commonly used luciferase enzymes are Renilla luciferase (RLuc), RlucII, Rluc8 (a mutant form of Renilla luciferase), firefly luciferase, Oplophorus luciferase (OLuc), and NanoLuc® (a mutant form of OLuc (Promega)). Other suitable luminescence donors are known to those of ordinary skill in the art.

In the methods provided herein, BRET fluorescence acceptors may include any suitable fluorophore that meets the criteria for a BRET fluorophore as discussed herein (i.e., meet the conditions for BRET to occur when in sufficiently close proximity to a particular luminescence donor). Exemplary fluorophores that may be used as donor and/or acceptor fluorophores include but are not limited to, cyanine dyes (e.g., Cy2, Cy3, Cy3B, Cy5, Cy5.5, Cy7, etc.), Alexa Fluor (AF) dyes (e.g., AF 647, AF 555, or AF 488), rhodamine dyes (e.g., fluorescein, FITC, Texas Red, ROX), ATTO dye (e.g., ATTO 532 or 655), fluorescent proteins such as green fluorescent protein (GFP), yellow fluorescent proteins (e.g., YFP, Citrine, Venus, and Ypet), cyan fluorescent protein (ECFP, Cerulean, CyPet, mTurquoise2) or photoactivabale fluorescent proteins, such as PAGFP, PSCFP, PSCFP2, Dendra, Dendra2, EosFP, tdEos, mEos2, mEos3, PAmCherry, PAtagRFP, mMaple, mMaple2, and mMaple3. Other suitable fluorophores are known to those of ordinary skill in the art.

In some embodiments of the BRET assays described herein, one protein of interest is tagged with a bioluminescent energy donor (e.g., luciferase from Renilla reniformis or Oplophorus gracilirostris), and the other protein is tagged with a fluorescent energy acceptor (e.g., GFP or YFP). When the two proteins are close together, in the presence of a suitable substrate (e.g., coelenterazine for luciferase), the bioluminescent energy donor causes the fluorescent energy acceptor to emit detectable fluorescence. Detection of this fluorescence indicates that the proteins are interacting. In some embodiments of the methods provided herein, either a GPCR of interest or a fusion protein described herein comprise a BRET luminescent donor. In some embodiments, a GPCR of interest comprising RLuc is expressed in cells and used in BRET assays. In some embodiments of the methods provided herein, either a GPCR of interest or a fusion protein described herein comprises a BRET fluorescence acceptor. In some embodiments, the fusion proteins used in BRET assays described herein comprise a YFP fluorophore BRET fluorescence acceptor. Methods similar to BRET (e.g., fluorescence resonance energy transfer (FRET) or biomolecular fluorescence complementation (BiFC)) may also be useful in the methods of the present disclosure.

In some embodiments, purified forms of a fusion protein described herein and at least one GPCR may be used in X-ray crystallography experiments. In some embodiments, the fusion protein is able to bind to the GPCR more strongly than the endogenous form of the arrestin polypeptide of the fusion protein, which may stabilize the interaction and facilitate X-ray crystallography.

Also provided herein is a method for identifying whether a drug compound impacts arrestin-mediated signaling. In some instances, the drug compound is a small molecule or peptide. As used herein, a drug compound “impacts” arrestin-mediated signaling when it alters or interferes with an aspect or aspects of arrestin-mediated signaling. In some instances, the drug compound can be an agonist. In some instances, the drug compound can be an antagonist. For example, as described above, one important aspect of arrestin-mediated signaling is recruitment of βarrestins to specific GPCRs upon activation of the GPCRs by agonists. A drug compound may impact arrestin-mediated GPCR signaling by blocking or amplifying this recruitment (e.g., by destabilizing or stabilizing the βarrestin-GPCR interaction). Any other observable change in arrestin-mediated GPCR signaling upon treatment with a drug compound may indicate that the drug compound impacts arrestin-mediated GPCR signaling. Another aspect of arrestin-mediated signaling that may be assessed using the methods herein is interaction between arrestin proteins and non-GPCR proteins (e.g., single transmembrane receptors, endocytic proteins, nuclear membrane proteins, etc.).

In some embodiments, the methods provided herein for identifying whether a drug compound impacts arrestin-mediated signaling comprise: (a) providing a plurality of cells that express a fusion protein described herein and one or more GPCRs, wherein the fusion protein is able to bind to and regulate the signaling of at least one of the GPCRs; (b) treating the plurality of cells with a drug compound, thereby forming a drug-treated plurality of cells; (c) assessing activation and/or signaling of the GPCR in the drug-treated plurality of cells; and (d) comparing the GPCR activation and/or signaling in the drug-treated plurality of cells to the GPCR activation and/or signaling assessed in a control plurality of cells that have not been contacted with the drug compound, wherein a difference in GPCR activation and/or signaling between the drug-treated plurality of cells and the control plurality of cells indicates that the drug compound impacts arrestin-mediated GPCR signaling of the GPCR.

In some embodiments, the methods provided herein for identifying whether a drug compound impacts arrestin-mediated signaling or activity comprise: (a) providing a plurality of cells that express a fusion protein described herein and one or more non-GPCR signaling proteins, wherein the fusion protein is able to bind to and regulate the signaling of at least one of the non-GPCR signaling proteins; (b) treating the plurality of cells with a drug compound, thereby forming a drug-treated plurality of cells; (c) assessing activation and/or signaling of the non-GPCR signaling protein in the drug-treated plurality of cells; and (d) comparing the non-GPCR signaling protein activation and/or signaling in the drug-treated plurality of cells to the non-GPCR signaling protein activation and/or signaling assessed in a control plurality of cells that have not been contacted with the drug compound, wherein a difference in non-GPCR signaling protein activation and/or signaling between the drug-treated plurality of cells and the control plurality of cells indicates that the drug compound impacts arrestin-mediated signaling.

In some embodiments, assessing activation and/or signaling of the GPCR and/or the non-GPCR signaling protein comprises detecting recruitment of an arrestin protein to the GPCR and/or the non-GPCR signaling protein, e.g., by detecting the subcellular localization pattern of the fusion protein (e.g., using any of the methods described above). In some embodiments, assessing activation and/or signaling of the GPCR and/or the non-GPCR signaling protein comprises detecting an interaction between an arrestin protein and the GPCR and/or the non-GPCR signaling protein, e.g., by detecting protein-protein interaction of the fusion protein and the GPCR and/or the non-GPCR signaling protein (e.g., using any of the methods described above).

In some embodiments, assessing activation and/or signaling of the GPCR and/or the non-GPCR signaling protein comprises any known GPCR and/or non-GPCR signaling protein functional assay. For example, a GPCR functional assay may include a receptor internalization assay, a βarrestin recruitment assay, or a label-free whole cell assay, as described, e.g., in Zhang et al. 2012. Acta Pharmacologica Sinica 33:327-384. In some embodiments, a GPCR and/or a non-GPCR signaling protein functional assay may include analysis of downstream effects of GPCR or non-GPCR signaling. For example, mass spectrometry may be used to evaluate changes in protein modifications (e.g., protein phosphorylation, ubiquitination, SUMOylation, etc.), or RNA sequencing may be used to evaluate GPCR-mediated or non-GPCR-mediated gene expression regulation. βarrestin-dependent signaling may impact cell motility, chemotaxis, cell viability, secretion of exosomes, and/or secretion of cytokines. As such, an assay for assessing any of these characteristics may be used to assess arrestin-mediated activation and/or signaling of a GPCR and/or a non-GPCR signaling protein. These assays include, but are not limited to, cell migration assays, chemotaxis assays, cell viability assays, cytotoxicity assays, exosome secretion assays, and cytokine secretion assays. In the context of this disclosure, these known assays can be performed using cells that express the fusion protein as described herein.

Any of the methods provided herein may be applied to any known or newly discovered GPCR (i.e., GPCR of interest). In some embodiments, the GPCR of interest in the methods provided herein is angiotensin type 1a receptor (AT₁ₐR) β₂ adrenergic receptor (β₂AR), D₂ dopamine receptor (D2R), β₁ adrenergic receptor (β₁AR), D₁ dopamine receptor (D1R), V₂ vasopressin receptor (V₂R) and/or glucagon receptor (GCGR). Additionally, any of the methods that may be applied to one GPCR may also be applied to multiple GPCRs. For example, a fusion protein described herein may be expressed in a population of cells that express one GPCR of interest, two GPCRs of interest, or more.

In some embodiments, cells that express one or more GPCRs are cells that endogenously express the GPCR(s), i.e., the cellular genomes comprise a gene or genes encoding the GPCR(s) and the gene is expressed when the cells are used in the methods described herein. In some embodiments, any of the methods described above for introducing a fusion protein into a host cell may be used to introduce one or more GPCRs of interest into cells. In some embodiments, a recombinant nucleic acid, DNA construct, or vector comprising one or more genes encoding one or more GPCRs is introduced into cells. In some embodiments, the cells express the fusion protein before introduction of the one or more GPCRs of interest. In some embodiments, the one or more GPCRs of interest are introduced into cells before expressing the fusion protein. The GPCRs described herein may comprise any of the polypeptide modifications described above (e.g., detectable moieties, affinity tags, etc.). In some embodiments, GPCR polypeptide modifications are encoded in an exogenous transgene that is introduced into cells for use in the methods herein. In some embodiments, CRISPR/Cas9 editing may be used to modify endogenously expressed GPCRs for use in the methods herein.

In some embodiments, cells that express one or more non-GPCR signaling proteins are cells that endogenously express the non-GPCR signaling protein(s), i.e., the cellular genomes comprise a gene or genes encoding the non-GPCR signaling protein(s) and the gene is expressed when the cells are used in the methods described herein. In some embodiments, any of the methods described above for introducing a fusion protein into a host cell may be used to introduce one or more non-GPCR signaling proteins of interest into cells. In some embodiments, a recombinant nucleic acid, DNA construct, or vector comprising one or more genes encoding one or more non-GPCR signaling proteins is introduced into cells. In some embodiments, the cells express the fusion protein before introduction of the one or more non-GPCR signaling proteins of interest. In some embodiments, the one or more non-GPCR signaling proteins of interest are introduced into cells before expressing the fusion protein. The non-GPCR signaling proteins described herein may comprise any of the polypeptide modifications described above (e.g., detectable moieties, affinity tags, etc.). In some embodiments, non-GPCR signaling protein polypeptide modifications are encoded in an exogenous transgene that is introduced into cells for use in the methods herein. In some embodiments, CRISPR/Cas9 editing may be used to modify endogenously expressed non-GPCR signaling proteins for use in the methods herein.

In some embodiments, the methods provided herein may comprise treatment of a plurality of cells expressing a fusion protein described herein with at least one agonist compound that activates one or more GPCRs expressed by the plurality of cells. In some embodiments, the methods may comprise treatment of a plurality of cells expressing a fusion protein described herein with at least one antagonist compound that inhibits the activity of one or more GPCRs expressed by the plurality of cells. Any agonist compound known to activate a GPCR of interest or antagonist compound known to inactivate a GPCR of interest may be used. Agonist compounds may include, for example, isoproterenol (Iso), dopamine, arginine-vasopressin (AVP), glucagon, or any other known GPCR agonist. Antagonist compounds may include, for example, carvedilol, propranolol, a beta blocker compound, a vaptan compound, or any other known GPCR antagonist. In some embodiments, the methods provided herein may comprise treatment of a plurality of cells expressing a fusion protein described herein with any other type of compound that modulates the function of one or more GPCRs of interest (e.g., an allosteric modulator, a biased ligand, etc.) See, e.g., Sum et al. Pharmacological Characterization of GPCR Agonists, Antagonists, Allosteric Modulators and Biased Ligands from HTS Hits to Lead Optimization. 2019 Nov. 1. In: Markossian et al., ed. Assay Guidance Manual. Bethesda (Md.): Eli Lilly & Company and the National Center for Advancing Translational Sciences; 2004. Available from: ncbi.nlm.nih.gov/books/NBK549462/.

In some embodiments, the plurality of cells is treated with a GPCR agonist compound and/or antagonist compound prior to detecting the localization pattern of the fusion protein or the GPCR in the plurality of cells. In some embodiments, the plurality of cells is treated with a GPCR agonist compound and/or antagonist compound prior to detecting protein-protein interactions of the fusion protein and the GPCR in the plurality of cells. In some embodiments, the plurality of cells is treated with a GPCR agonist compound and/or antagonist compound prior to assessing activation and/or signaling of the GPCR in the plurality of cells. In some embodiments, the methods provided herein comprise comparison of GPCR activation and/or signaling in a drug-treated plurality of cells to GPCR activation and/or signaling in a control (i.e., non-drug-treated) plurality of cells. In such instances, either the drug-treated plurality of cells, the control plurality of cells, or both the drug-treated plurality of cells and the control plurality of cells may be treated with a GPCR agonist compound and/or antagonist compound prior to assessing GPCR activation and/or signaling. In some embodiments, a plurality of cells is treated with a GPCR agonist compound and/or antagonist compound prior to treatment of the plurality of cells with a drug compound.

In some embodiments, the methods provided herein may comprise treatment of a plurality of cells expressing a fusion protein described herein with at least one agonist compound that activates one or more non-GPCR signaling proteins (e.g., a single transmembrane cell receptor, a non-receptor protein, an endocytic protein, a nuclear membrane protein, etc.) expressed by the plurality of cells. In some embodiments, the methods may comprise treatment of a plurality of cells expressing a fusion protein described herein with at least one antagonist compound that inhibits the activity of one or more non-GPCR signaling proteins expressed by the plurality of cells. Any agonist compound known to activate a non-GPCR signaling protein of interest or antagonist compound known to inactivate a non-GPCR signaling protein of interest may be used. In some embodiments, the methods provided herein may comprise treatment of a plurality of cells expressing a fusion protein described herein with any other type of compound that modulates the function of one or more non-GPCR signaling proteins of interest (e.g., an allosteric modulator, a biased ligand, etc.).

In some embodiments, the plurality of cells is treated with a non-GPCR signaling protein agonist compound and/or antagonist compound prior to detecting the localization pattern of the fusion protein or the non-GPCR signaling protein in the plurality of cells. In some embodiments, the plurality of cells is treated with a non-GPCR signaling protein agonist compound and/or antagonist compound prior to detecting protein-protein interactions of the fusion protein and the non-GPCR signaling protein in the plurality of cells. In some embodiments, the plurality of cells is treated with a non-GPCR signaling protein agonist compound and/or antagonist compound prior to assessing activation and/or signaling of the non-GPCR signaling protein in the plurality of cells. In some embodiments, the methods provided herein comprise comparison of non-GPCR signaling protein activation and/or signaling in a drug-treated plurality of cells to non-GPCR signaling protein activation and/or signaling in a control (i.e., non-drug-treated) plurality of cells. In such instances, either the drug-treated plurality of cells, the control plurality of cells, or both the drug-treated plurality of cells and the control plurality of cells may be treated with a non-GPCR signaling protein agonist compound and/or antagonist compound prior to assessing non-GPCR signaling protein activation and/or signaling. In some embodiments, a plurality of cells is treated with a non-GPCR signaling protein agonist compound and/or antagonist compound prior to treatment of the plurality of cells with a drug compound.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

The following examples are offered to illustrate, but not to limit the claimed invention. Many of the following examples are further described in Nagi et al. 2020. Cellular Signaling 75:109759, which is hereby incorporated by reference in its entirety.

### Example 1: Materials and Methods Used in Examples

Antibodies. The antibodies used in the Examples herein include: anti-Flag M2 (Sigma: F3165), anti-HA 12CA5 (Roche: 11666606001), anti-β₂AR H20 (Santa Cruz: sc-569), anti-GAPDH-HRP (Cell Signaling: 3683), and anti-GFP/GFP-variants (MBL-598). Alexa 594 conjugated secondary antibody was obtained from Invitrogen. For immunoprecipitation of YFP-fusion proteins, GFP Monoclonal Antibody (3E6), A-11120, from Thermo Fisher Scientific, was used. Horseradish peroxidase-conjugated secondary antibodies were from GE/Amersham or Rockland Immunochemicals. Also used were Anti-Flag M2 affinity gel (Sigma: A2220), Protein G Plus/Protein A-Agarose (Calbiochem: IP10), (−)-Isoproterenol (Sigma: I2760), Dopamine (Sigma: H8502), Angiotensin II (Sigma: A9525), Arginine vasopressin (Sigma: V9879), N-Ethylmaleimide (Sigma: E1271), Triton X-100 (Sigma: T-9284), Piercer™ anti-HA magnetic beads (Thermo Fisher Scientific: 88837) and, DSP (dithiobis(succinimidyl propionate)) (Thermo Fisher Scientific: 22585). Lipofectamine 2000 was from Invitrogen.

Plasmids. The plasmid constructs, pcDNA3/mYFP-βarrestin2-K296R, pcDNA3/mYFP-βarrestin2-SUMO1, and pCDNA3-Flag-βarrestin2-SUMO1 were generated by standard cloning and/or mutagenesis protocols. The plasmid constructs, pcDNA3/mYFP-βarrestin2 and pcDNA3/mYFP-βarrestin2-ubiquitin have been reported before. See Jean-Charles et al. 2016. J Biol Chem 291:7450-7464. β₂AR-Rluc, was generously provided by Dr. Robert J. Lefkowitz. V₂R-RlucII was kindly provided by Dr. Michel Bouvier and HA-V₂R, HA-D2R, and D2R-RlucII were kindly provided by Dr. Marc Caron. YFP-SUMO1 plasmid was purchased from Addgene (#13380). See Ayaydin and Dasso. 2004. Mol Biol Cell 15:5208-5218.

Cell culture and transfection. Human Embryonic Kidney 293 (HEK-293) cells purchased from American Type Culture Collection (Manassas, Va.) were maintained in Minimum Essential (MEM) medium containing 10% fetal bovine serum and 100 μg/ml penicillin/streptomycin at 37° C. in a humidified incubator at 5% CO₂. Transfections were performed using Lipofectamine 2000 reagent (Invitrogen). HEK-293 cells stably transfected with Flag-β₂AR used in these studies have been described previously (25). HEK-293 cells with stable expression of HA-AT₁ₐR, HA-D2R, or HA-V₂R were generated by standard procedures using antibiotic selection as reported before. See Shenoy et al. 2006. J Biol Chem 281:1261-1273.

Human embryonic kidney 293T (HEK-293T) cells were cultured in Dulbecco's modified Eagle's medium (DMEM) supplemented with 4.5 g/L glucose, 10% fetal bovine serum and 1% Antibiotic-Antimycotic (Gibco) at 37° C. in a humidified atmosphere at 95% air and 5% CO₂. For transient expression of recombinant proteins, HEK-293T cells were seeded at a density of 6×10⁵ cells in 35 mm cell culture dishes such that they reach 40-50% confluence on the next day. Cells were then cultured for 24 h and transfected with vectors encoding BRET constructs as detailed below using calcium phosphate according to a previously published protocol. See Nagi and Shenoy. 2019. Methods Mol Biol 1957:93-104. HEK-293T cells are the preferred model system for BRET assays because of their high transfection efficiency of GPCR-Rluc constructs.

BRET assays. For assessing βarrestin2 (or βarrestin1) recruitment to receptors, bioluminescence resonance energy transfer (BRET) assays were performed in HEK-293T cells. Titration assays were first completed, which allowed determination of the specificity of association among different interaction partners. See Nagi and Shenoy. 2019, supra; Gales et al. 2005. Nature methods 2:177-184; Nagi et al. 2015. Cellular and molecular life sciences: CMLS 72:3543-3557; Audet and Pineyro. 2011. Methods in molecular biology 756:149-163; and Rebois et al. 2006. Journal of cell science 119:2807-2818. To achieve this, a fixed amount of the donor-tagged (Renilla luciferase, Rluc) construct was co-transfected with increasing amounts of the corresponding interaction partner bearing the acceptor (YFP). Donor-acceptor DNA ratios corresponding to the beginning of the saturation plateau were subsequently used for single-point and dose-response assays. See Richard-Lalonde et al. 2013. Molecular pharmacology 83:416-428 and Hamdan et al. 2006. Curr Protoc Neurosci Ch. 5, Unit 5:23. Two days after transfection, HEK-293T cells expressing different BRET pairs were washed with serum-free clear MEM media (Gibco) and exposed to vehicle or stimulated for the indicated times at 37° C. BRET readings were acquired using Synergy Neo2 plate reader and obtained 5 min after manual addition of Rluc substrate, coelenterazine h (Promega) to a final concentration of 5 μM.

The BRET signal generated was determined by calculating the ratio of light emitted by YFP (520-550 nm) over the light emitted by Rluc (440-480 nm). These values were then corrected by subtracting the background signal (detected when the Rluc-tagged construct was expressed without acceptor) from the BRET signal detected in cells co-expressing both donor and acceptor constructs. Agonist-induced BRET values were calculated by subtracting net BRET values of non-stimulated conditions from net BRET values corresponding to the stimulated conditions.

Immunostaining and confocal microscopy. In experiments assessing βarrestin2 (or βarrestin1) recruitment to receptors, HEK-293 stably expressing Flag-β₂AR, HA-D2R, HA-V₂R, or HA-AT₁ₐR were seeded on poly lysine-coated 35-mm glass bottom plates at a density of 100,000 cells per dish. On the following day, cells were transiently transfected with 500 ng of YFP-tagged constructs (YFP-βarrestin2-WT, YFP-βarrestin2-K296R, YFP-βarrestin2-SUMO1, YFP-βarrestin2-Ub, or YFP-SUMO1) using Lipofectamine 2000 (Invitrogen). Twenty-four hours post-transfection, cells were serum-starved for 4 h, and either left untreated or treated with respective agonists for the indicated times in the Figure descriptions. After stimulation, cells were fixed with 5% formaldehyde diluted in phosphate-buffered saline (PBS), permeabilized with 0.1% Triton in PBS containing 2% bovine serum albumin for 20 min and incubated at room temperature with appropriate primary antibody. The next day, cells were washed three times with PBS and incubated with Alexa594 conjugated secondary antibody for 1 h, followed by repeated washes using PBS. Confocal images were acquired using Zeiss LSM510 laser-scanning microscope using multitrack sequential with excitation (488 and 568 nm) and emission (BP 505-550 nm and LP 585 nm) filter sets. Images (1024×1024 pixels) were collected using either a 63× or 100× oil immersion lens. Images were processed for Figure presentation using Adobe Photoshop software, and any increase or decrease of brightness/contrast was applied to the entire image. The nuclear rim localization of YFP-βarrestin2-SUMO1 is readily visualized in cells with low to moderate expression of YFP-protein. In cells with high expression, this localization is present, and can be discerned by enlarging sections of the nuclear rim.

Cross-linking, Immunoprecipitation and Immunoblotting. HEK-293 cells stably expressing Flag-β₂AR, HA-D2R, or HA-V₂R were transiently transfected with 2 μg of YFP-βarrestin2-WT or YFP-βarrestin2-SUMO1 using Lipofectamine 2000 (Invitrogen). Twenty-four hours post-transfection, cells were starved in PBS containing 10 mM HEPES (pH 7.4) for 1 h and stimulated with vehicle or agonist (isoproterenol, 1 μM; or AVP 1 μM, or Dopamine 1 μM) at 37° C. for desired times. After stimulations, β₂AR stable cells were treated with the crosslinker DSP to a final concentration of 480 nM, and plates were rocked for 20 min at room temperature. Reaction was quenched by adding 25 μl of 1M Tris-Cl pH 8.5 per 1 mL volume of buffer in the dish and rocked for additional 5 min at room temperature. For the HA-D2R and HA-V₂R samples, detection of protein association with βarrestin2 did not require chemical crosslinking.

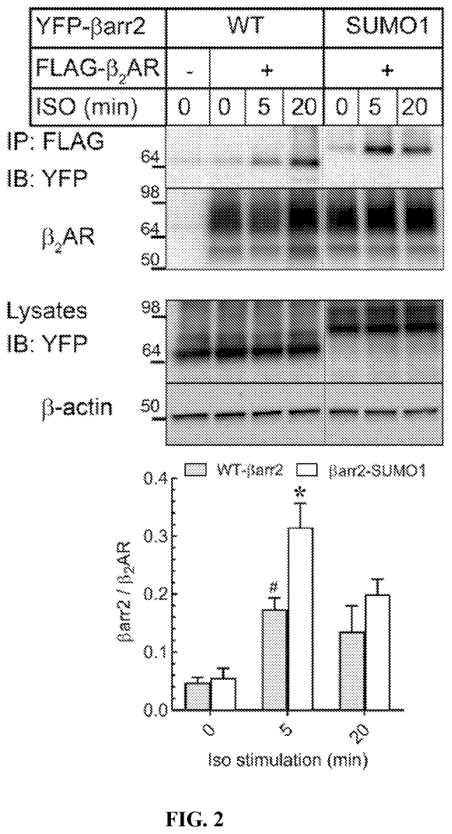

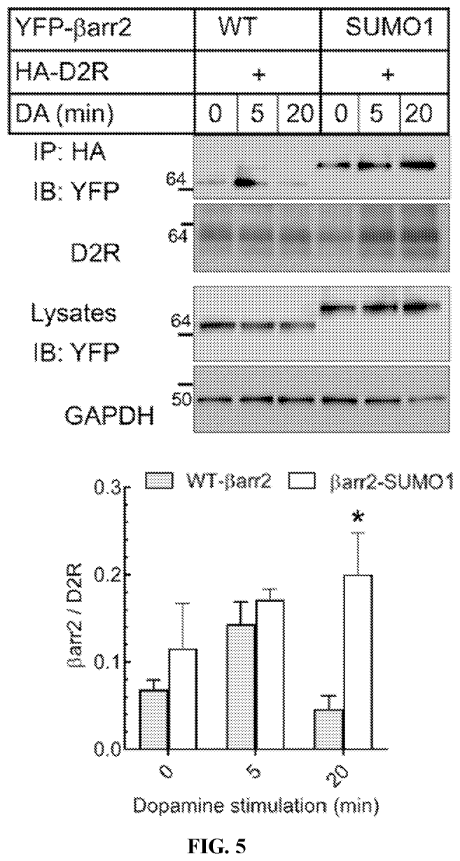

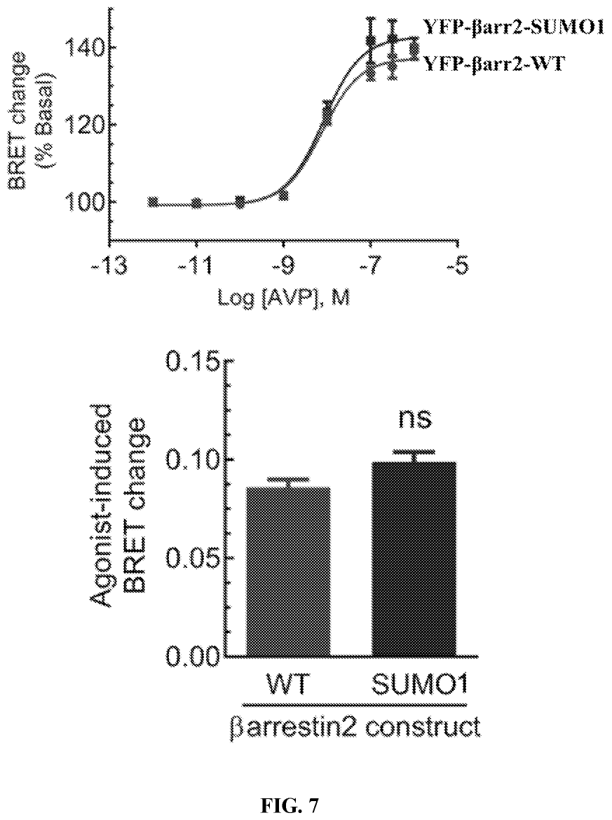

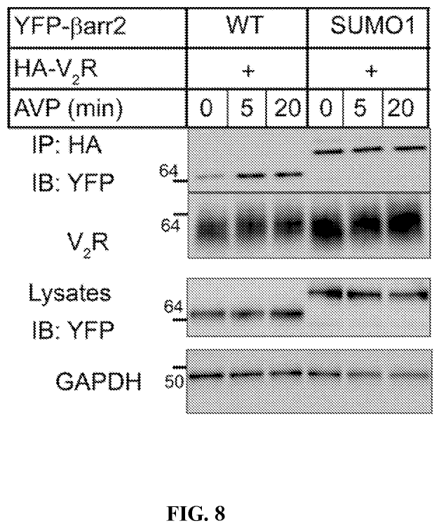

Harvested cells were washed with ice-cold PBS (pH 7.4) and solubilized in an ice-cold lysis buffer (50 mM HEPES (pH 7.5), 2 mM EDTA, 250 mM NaCl, 10% (v/v) glycerol, and 0.5% (v/v) IGEPAL CA-630) that was supplemented with phosphatase and protease inhibitors (1 mM sodium orthovanadate, 1 mM sodium fluoride, 1 mM phenylmethylsulfonyl fluoride, 5 μg/ml leupeptin, 5 μg/ml aprotinin, 1 μg/ml pepstatin A, and 100 μM benzamidine). Cell lysates were centrifuged at 13,000 rpm for 20 min at 4° C. to remove cell debris and the supernatant containing membranes and cytosol was recovered. Cell lysate protein concentrations were determined by Bradford protein assay and equivalent μg of proteins were immunoprecipitated using anti-FLAG M2 antibody resin (for FIG. 2) or Anti-HA magnetic beads from Thermo Pierce (for FIG. 5, FIG. 7, and FIG. 8). Samples were incubated overnight at 4° C., then washed three times with lysis buffer to eliminate non-specific proteins, and proteins bound to beads were eluted in SDS-PAGE sample buffer. Samples separated on SDS-PAGE were transferred onto nitrocellulose (Bio-Rad), and YFP-tagged βarrestin2 fusion proteins associated with Flag-β₂AR, HA-D2R or HA-V₂R were revealed using anti-GFP antibody (MBL-598) and anti-rabbit secondary HRP-conjugated antibody (1:3000; GE Healthcare). Flag-β₂ARs in each sample were detected by probing membranes with anti-β₂AR H-20 antibody (1:1000; Santa Cruz Biotechnology) and the corresponding secondary antibody (1:5000; GE Healthcare). HA-D2R or HA-V₂R were detected using an anti-HA IgG (Cell Signaling Technologies). Chemiluminescence detection reagents (SuperSignal West Pico reagent, Pierce) were used to reveal the blotted proteins, and relative intensities of the labeled bands were detected by densitometric scanning using a charge-coupled device camera system (Bio-Rad Chemidoc-XRS) and quantified with Image-Lab software (Bio-Rad).

siRNA transfection and immunoblotting. HEK-293 or HEK-293T cells stably expressing the β₂AR were transfected with either non-targeting control siRNA or siRNA targeting βarr2 purchased from Dharmacon GE Healthcare Life Sciences as described previously. See Luttrell et al. 2018, supra. Early passage cells on 6-well dishes at a confluence of 40-50% were transfected with 3.5 ug siRNA using Lipofectamine 2000™ in serum-free media. After 4 h, complete media was added to the transfected cells, and the cells were then grown for 48 h at 37° C. before conducting assays. Cells were serum starved for 1 h prior to stimulation with 1 μM isoproterenol for 20 min. After stimulation, cells were solubilized by adding 2×-SDS-sample buffer, followed by disruption by sonication. Equal amount of cell lysates were resolved on 10% SDS-polyacrylamide gels (ProtoGel, National Diagnostics). RanGAP1, βarrestins and GAPDH were detected by immunoblotting with rabbit monoclonal anti-RanGAP1 antibody (Abcam ab92360, 1:1000), anti-βarrestin (A1CT, 1:3,000) and rabbit monoclonal anti-GAPDH (HRP conjugate, CST 3683, 1:1000) respectively.

### Example 2: Disruption of the Consensus SUMOylation Site in βArrestin2 does not Alter its Plasma Membrane Translocation and Interaction with Activated GPCRs

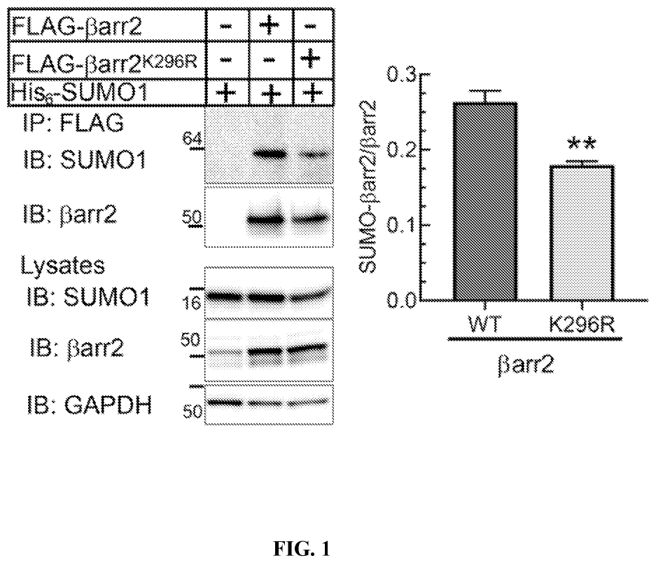

SUMOylation is targeted to a canonical protein sequence (Ψ-K-X-D/E), where Ψ is an aliphatic amino acid, K is the target site for the covalent modification by SUMO, X is any amino acid and is followed by an acidic residue. See Sampson et al. 2001. J Biol Chem 276:21664-21669. Previous studies have shown that lysine-296 in bovine and human βarrestin2 serves as a target site for SUMOylation and mutation of lysine-296 did not eliminate but significantly reduced SUMO conjugation of βarrestin2. See Wyatt et al. 2011 and Xiao et al. 2015, both supra. To test whether this is also true for rat βarrestin2, SUMOylation of rat βarrestin2 was compared to that of βarrestin2-K296R overexpressed in HEK-293 cells along with His-SUMO1 (FIG. 1). SUMOylation was significantly decreased but not eliminated in βarrestin2-K296R, suggesting the possibility that lysines in βarrestin2 that do not conform to the consensus motif may be targeted for SUMOylation.

Whether or not site-specific SUMOylation at lysine 296 impacts βarrestin2 trafficking and localization in cells was tested next. Trafficking and association of βarrestin2 and βarrestin2-K296R with activated β₂AR, D2R, AT₁ₐR and V₂R were compared in HEK-293 cells (data not shown). With these GPCRs, similar patterns of sub-cellular distribution of YFP-βarrestin2 and YFP-βarrestin2-K296R were observed in quiescent cells. Upon agonist activation, transient plasma membrane translocation of both βarrestin2 as well as βarrestin2-K296R to the β₂AR and D2R was observed. Robust endosomal localization of AT₁ₐR-βarrestin2 and V₂R-βarrestin2 complexes, for both WT and K296R βarrestins, was also observed. Accordingly, it is inferred that the SUMOylation at the consensus motif in βarrestin2 has negligible effect of the overall trafficking and GPCR affinity of βarrestin2. In fact, to eliminate SUMOylation of bovine βarrestin2, a total of four lysines had to be mutated, and only two of them were within a canonical SUMO motif. Nonetheless, the 4KR-arrestin3 mutant still translocated and associated with the activated β₂ARs at the plasma membrane. See Wyatt et al. 2011, supra. Accordingly, either SUMOylation of lysine(s) in non-canonical sequence motif or ubiquitination of βarrestin2 may overcome the defect produced by the disruption of SUMOylation at the canonical site.

### Example 3: Subcellular Distribution of βArrestin2-SUMO1 Fusion Protein

According to published studies, βarrestin2 binds the de-SUMOylase SENP1 (see Xiao et al. 2015, supra), and hence deducing the impact of SUMOylation on βarrestin2 trafficking could be elusive due to the dynamic nature of the modification. To circumvent this issue, a YFP tagged βarrestin2-SUMO1 fusion protein was generated, which would be resistant to the enzymatic activity of SENP1. YFP-βarrestin2-SUMO1 expressed in the cytoplasm akin to YFP-βarrestin2; but was also detected at the nuclear membrane (data not shown). YFP-SUMO1 was predominantly nuclear, and did not display the ring like distribution observed with βarrestin2-SUMO1 (data not shown). The distribution of mYFP-βarrestin2-SUMO1 (85 cells) versus mYFP-SUMO1 (80 cells) was observed, and while all cells expressing βarrestin2-SUMO1 showed localization of YFP fluorescence at the nuclear membrane, none of the cells transfected with YFP-SUMO1 showed this pattern. Previous studies detected YFP-SUMO1 mostly in the nucleus and nucleolus, along with punctate pattern at the nuclear membrane in HeLa cells that were subjected to mitotic synchronization (see Ayaydin and Dasso. 2004, supra); however, no distinct localization of YFP-SUMO1 was observed at the nuclear membrane in HEK-293 cells. In addition, fusion of the cytoplasmic protein pyruvate kinase with SUMO1 did not localize pyruvate kinase to the nuclear membrane. See Matunis et al. 1998. J Cell Biol 140:499-509. While YFP-SUMO1 was concentrated in the nucleus, and nucleoli, very little βarrestin2-SUMO1 was detected in the nucleus or nucleolus. Thus, the subcellular distribution of βarrestin-SUMO1 is different compared with YFP-SUMO1, and the difference is not due to trafficking properties of SUMO1 itself, but rather represents the properties of the βarrestin2-SUMO1 fusion protein and might mimic the localization of persistently SUMOylated βarrestin2. Furthermore, although ubiquitin and SUMO1 have high structural homology, βarrestin-ubiquitin fusion protein was undetectable at the nuclear membrane (data not shown).

### Example 4: βArrestin2-SUMO1 Fusion Protein Associates with Agonist-Activated β₂AR, β₁AR, and D1R with Higher Affinity than βArrestin2

The trafficking of βarrestin2-SUMO1, and βarrestin2, was analyzed in HEK-293 cells with stable expression of the β₂AR. In quiescent cells, minimal colocalization of βarrestin2 with the β₂AR was observed; upon agonist activation, translocation of βarrestin2 to the β₂AR at the plasma membrane was observed after 5 minutes of agonist stimulation (data not shown). However, after 20 minutes of agonist activation, βarrestin2 and β₂AR dissociate from each other, and no colocalization was detected. For βarrestin2-SUMO1, plasma membrane translocation was detected upon β₂AR agonist activation, and, intriguingly, βarrestin2-SUMO1 was detected in endocytic vesicles. These β₂AR-βarrestin2-SUMO1 complexes persisted with longer agonist activation (data not shown). Confocal images were taken of cells stably expressing the β₂AR and transiently expressing YFP-SUMO1 (data not shown). In both quiescent and agonist-treated cells, the SUMO1 protein remained in the nucleus. SUMO1 and Ub share the same structural properties, and previously βarrestin2-Ub fusion protein demonstrated a robust binding and endosomal colocalization with the β₂AR. See Shenoy et al. 2007, supra; Shenoy and Lefkowitz. 2005. J Biol Chem 280:15315-15324; and Shenoy and Lefkowitz. 2003. J Biol Chem 278:14498-14506. Therefore, the endosomal trafficking was compared for βarrestin2-ubiquitin (βarrestin2-Ub) fusion protein and internalized β₂ARs. The magnitude of colocalization of internalized β₂ARs and βarrestin2-Ub was much greater than colocalization of βarrestin2-SUMO1 and β₂AR. Nonetheless, an accelerated mobilization of β₂AR and βarrestin2-SUMO1 in endosomal vesicles was observed compared to βarrestin2-Ub.

The association of βarrestin2-SUMO1 with β₂AR complexes was also observed using chemical crosslinking (see Example 1) (see Shenoy et al. 2007, supra) and co-immunoprecipitation (FIG. 2). HEK-293 cells stably expressing FLAG-β₂AR were used, with transient expression of βarrestin2 or βarrestin2-SUMO1. The receptors were immunoprecipitated under nonstimulated or stimulated conditions (5 and 20 min, 1 μM isoproterenol), and βarrestin2 or βarrestin2-SUMO1 were detected by Western blotting (FIG. 2, top panel). βarrestin2-SUMO1 binds to activated receptors approximately 2-3 fold more strongly than the WT βarrestin2 at 5 minutes of isoproterenol stimulation (FIG. 2, bottom panel). Accordingly, βarrestin2-SUMO1 has an increased affinity for agonist-activated β₂ARs.

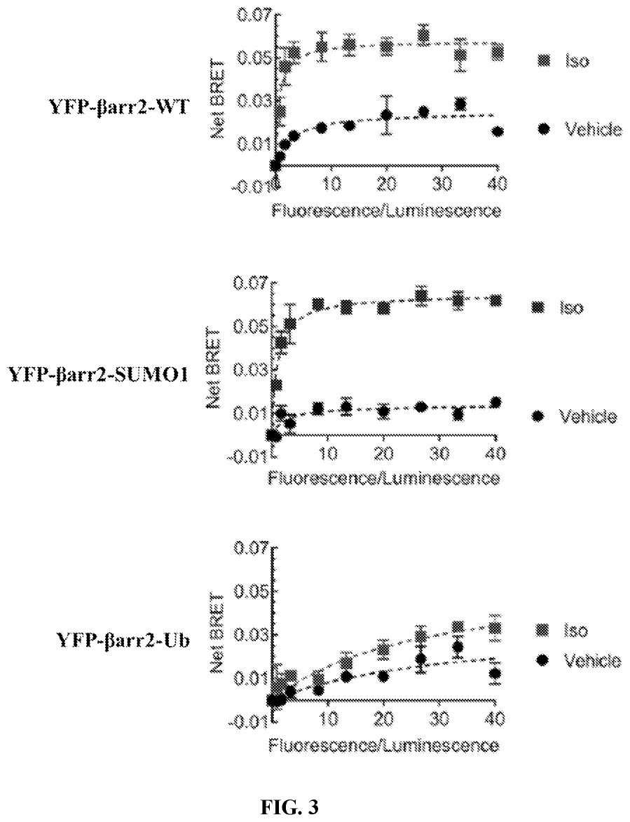

Whether or not the recruitment of βarrestin2 fusion proteins to the β₂AR can be measured in living cells was assessed using the BRET-based proximity assay. In this approach, titrations curves were used in which HEK-293T cells were transiently transfected with a fixed amount of donor-tagged receptor subunits genetically fused to Renilla Luciferase (β₂AR-Rluc) and increasing amounts of YFP βarrestin2 acceptor constructs (YFP-βarrestin2, YFP-βarrestin2-SUMO1 or YFP-βarrestin2-Ub) (FIG. 3). Cells were then treated for 5 min with isoproterenol (1 μM) and BRET measurements followed immediately afterward. Minimal BRET signal was detected in cells that were not exposed to the agonist, which could be caused by random collisions between energy donor and acceptors or from negligible constitutive βarrestin2 recruitment. See Mercier et al. 2002. J Biol Chem 277:44925-44931. However, when exposed to the agonist, increasing concentrations of the acceptor construct induced a progressive increase in BRET signal until increments became minimal. This saturable increase in energy transfer at βarrestin2/β₂AR interfaces indicates that all fusion versions of the regulatory protein were specifically recruited to the receptor.

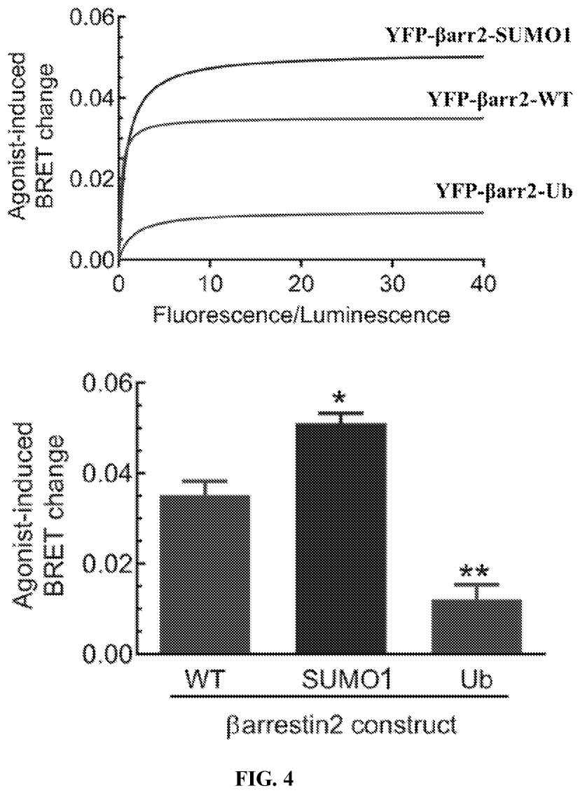

The specific ligand-induced βarrestin2 recruitment effects were calculated by subtracting the BRET signal observed in control cells from the total signal obtained when exposed to the agonist (FIG. 4). For the different pairs tested, YFP-βarrestin2-SUMO1 showed the highest change in the net BRET value for the protein interaction with β₂AR (FIG. 4, bottom panel) among βarrestin2 constructs. Taken together with the confocal and immunoprecipitation analyses, it is inferred that YFP-βarrestin2-SUMO1 adopts a conformation that is more suitable to interact with the C-terminus of the β₂AR than YFP-βarrestin2. On the other hand, although YFP-βarrestin2-Ub shows robust recruitment to the membrane and colocalization with the receptor after 5 min agonist-stimulation in confocal experiments, and in co-immunoprecipitation analyses (see Shenoy et al. 2007, and Shenoy and Lefkowitz. 2003, both supra) it showed the lowest ligand-induced BRET signal. Therefore, although YFP-βarrestin2-Ub fusion protein associates tightly with the β₂AR (see Shenoy et al. 2007, Shenoy and Lefkowitz. 2005, Shenoy and Lefkowitz. 2003, all supra, and Shenoy and Lefkowitz. 2005. Nat Cell Biol 7:1159-1161), it is likely that the orientation of YFP-βarrestin2-Ub poises it in a conformation sub-optimal for BRET. See Hamdan et al. 2006, supra. Alternatively, the reciprocal patterns observed for BRET for the βarrestin2-SUMO′ (increase in BRET) and βarrestin2-Ub (decrease in BRET) compared with βarrestin2 also suggests the existence of a specific but different ligand-induced conformation for each of the βarrestin2 fusion proteins.

The trafficking of βarrestin2-SUMO1, and βarrestin2, was also analyzed in HEK-293 cells with stable expression of the β₁ adrenergic receptor (β₁AR), using similar methods to those described above for β₂AR. In quiescent cells, confocal images showed minimal colocalization of βarrestin2 and β₁AR; upon activation with the agonist isoproterenol for 5 minutes, βarrestin2 and β₁AR show colocalization at the plasma membrane. At longer duration of agonist stimulation, β₁AR and βarrestin2 complexes fall apart due to the internalization of β₁AR into endosomal vesicles (data not shown). Similar confocal images were obtained with a βarrestin2-SUMO1 fusion protein in HEK-293 cells with stable expression of β₁AR (data not shown). β₁AR associated with βarrestin2-SUMO1 strongly and stably such that both proteins internalized together and formed complexes at endocytic vesicles.

The trafficking of βarrestin2-SUMO1, and βarrestin2, was also analyzed in HEK-293 cells with stable expression of the D₁ dopamine receptor (D1R), using similar methods to those described above for β₂AR. In quiescent cells, confocal images showed minimal colocalization of βarrestin2 and D1R; upon activation with the agonist dopamine for 15 minutes, βarrestin2 and D1R show weak association at the plasma membrane (data not shown). Similar confocal images were obtained with a βarrestin2-SUMO1 fusion protein in HEK-293 cells with stable expression of D1R (data not shown). D1R associated with βarrestin2-SUMO1 strongly and stably such that both proteins formed complexes at endocytic vesicles.

### Example 5: βArrestin2-SUMO1 Shows Enhanced Affinity with the D2R but not with the V₂R

The specificity of the observations in the previous Examples of βarrestin2-SUMO1 interaction with the β₂AR were established by assessing whether βarrestin2-SUMO1 could interact with other GPCRs in an enhanced capacity when compared with βarrestin2. The D₂ dopamine receptor (D2R), which possesses a similar trafficking ‘Class A’ plasma membrane recruitment of βarrestin2, like the β₂AR, and the V₂R, which has a very high affinity for βarrestin2 association were tested for interaction with βarrestin2-SUMO1. The D2R leads to activation of the inhibitory G protein (Gi) (see Beaulieu and Gainetdinov. 2011. Pharmacological reviews 63:182-217 and Sibley et al. 1992. Trends in pharmacological sciences 13:61-69), recruits βarrestin2 transiently at the plasma membrane, and internalizes without bound βarrestin upon activation. See Peterson et al. 2015. Proc Natl Acad Sci USA 112:7097-7102. The V₂R couples to the stimulatory G protein (Gs) (see Erlenbach and Wess. 1998. J Biol Chem 273:26549-26558), but co-traffics with bound βarrestin2 and forms βarrestin2-ERK signaling complexes at endosomes. See Tohgo et al. 2003. J Biol Chem 278:6258-6267 and Oakley et al. 2000. J Biol Chem 275:17201-17210. Notably, because of its high affinity to βarrestins, V₂R C-tail residues are often appended to other GPCRs to stabilize βarrestin2-receptor complex. See Cahill et al. 2017. Proc Natl Acad Sci USA 114:2562-2567.

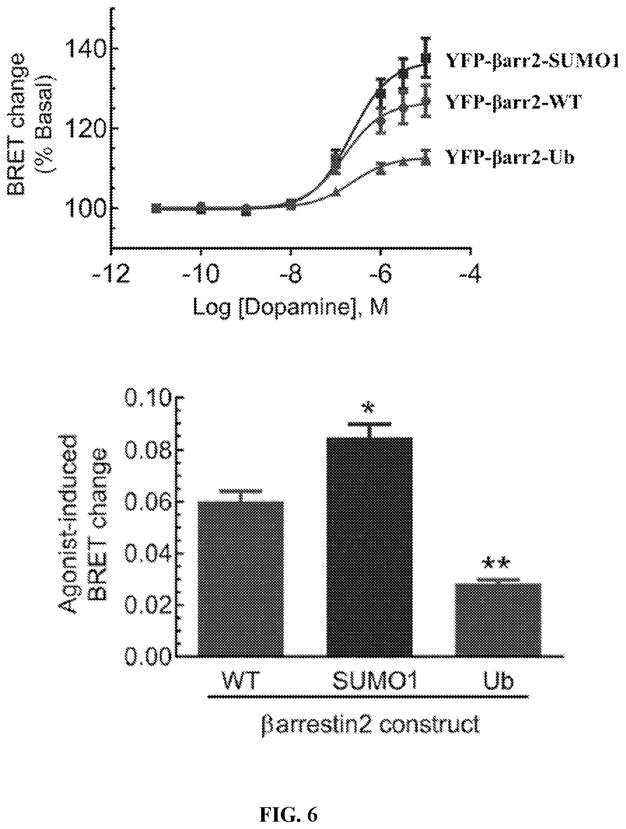

Agonist-induced βarrestin2 recruitment to the D2R was evaluated by confocal microscopy (data not shown), coimmunoprecipitation (FIG. 5) and BRET (FIG. 6) in the same manner as described for the β₂AR (FIGS. 2-4). In quiescent cells, the overall patterns of βarrestin2, βarrestin2-SUMO1, SUMO1, and βarrestin2-Ub sub-cellular distributions in HEK-293 cells stably expressing the D2R were identical to the patterns observed with the β₂AR expressing cells (data not shown). Although βarrestin2 translocates to the plasma membrane upon D2R activation, its interaction with the D2R is not stable. In contrast, both βarrestin2-SUMO1 and βarrestin2-Ub associate stably with the D2Rs localized on endocytic vesicles, along with internalized D2Rs. Substantial D2R internalization in endocytic vesicles was also observed in cells expressing βarrestin2-SUMO1 compared to cells expressing βarrestin2. On the other hand, there was no change in YFP-SUMO1 localization after D2R agonist-activation.

The association of βarrestin2-SUMO1 with D2R complexes was determined using co-immunoprecipitation assays (FIG. 5). HEK-293 cells stably expressing HA-D2R were used, with transient expression of βarrestin2 or βarrestin2-SUMO1. The receptors were immunoprecipitated under nonstimulated or stimulated conditions (5 and 20 min, 1 μM dopamine), and βarrestin2 or βarrestin2-SUMO1 were detected by Western blotting (FIG. 5, top panel). βarrestin2-SUMO1 binds to activated receptors approximately 3-4 fold more strongly than the wild-type βarrestin2 at 20 minutes of stimulation (FIG. 5, bottom panel), although no significant difference in D2R-binding affinity was observed between βarrestin2 and βarrestin2-SUMO1 at five minutes of agonist-stimulation. Thus, SUMOylation status of βarrestin2 can enable its persistent association with internalized D2Rs.

A concentration-dependent increase in BRET signals between Rluc-tagged D2R and YFP-tagged βarrestin2 constructs, corresponding to βarrestin2 recruitment to the receptor, was observed (FIG. 6). As detected for the β₂AR system, the agonist-mediated increase in energy transfer was greater with YFP-βarrestin2-SUMO1 than with YFP-βarrestin2 for the D2R; additionally, the net-BRET signal was only minimal for YFP-βarrestin2-Ub upon receptor stimulation (FIG. 6). The weak signals observed for D2R-βarrestin2-Ub association by BRET is attributed to its unfavorable conformation for BRET.

No significant differences were found between βarrestin2 and βarrestin2-SUMO1 in the pattern of sub-cellular colocalization with activated V₂Rs (data not shown), nor in the magnitude of interaction defined by BRET (FIG. 7). Concordantly, both βarrestin2 and βarrestin2-SUMO1 showed equivalent binding with agonist activated V₂Rs as determined by co-immunoprecipitation assays (FIG. 8). Constitutive interaction of YFP-βarrestin2-SUMO1 and HA-V₂Rs was also observed, which could be due to an increased binding affinity due to exposure of hydrophobic regions with detergent solubilization. Nonetheless, no difference was detected in binding affinity of βarrestin2 versus βarrestin2-SUMO1 with agonist activated V₂Rs by any of the approaches, namely sub-cellular distribution by confocal analyses, BRET, and coimmunoprecipitation. It is likely that, because activated and phosphorylated V₂R has a high affinity for βarrestins, as evidenced by the utilization of V₂R carboxyl tail residues to enhance βarrestin binding to GPCRs that have low affinity for βarrestins (see Oakley et al. 2000, supra and Evron et al. 2014. J Biol Chem 289:33442-33455), appending SUMO1 did not result in a further increase in V₂R-βarrestin2 association.

### Example 6: SUMOylation of βArrestin2 Facilitates its Protein Interactions at the Nuclear Membrane

Although βarrestins were discovered in the context of GPCR regulation, it is evident that they play a much broader role and regulate many types of receptors, and scaffold enzymes of the ubiquitination pathway and kinase phosphorylation cascades. See Peterson and Luttrell. 2017. Pharmacol Rev 69:256-297, Jean-Charles et al. 2016, supra, and Jean-Charles et al. 2016. J Cell Physiol 231:2071-2080. In HeLa cells subjected to mitotic synchronization, SUMO1 was localized at the nuclear membrane during interphase and at mitotic spindles during cell division, but the paralogs, SUMO2 and SUMO3, did not show these patterns of localization. This was attributed to the changes in RanGAP1 sub-cellular localization and the paralog-specific conjugation of RanGAP1 with SUMO1. See Ayaydin and Dasso. 2004, supra. Interestingly, RanGAP1 is one of the few SUMOylated proteins that does not localize in the nucleus, and SUMOylation of RanGAP1 is required, but not sufficient, for its localization at the nuclear envelope. Studies have also indicated that mere SUMO1 fusion to cytosolic proteins does not target them to locate to nuclear membranes, because fusion of SUMO1 to pyruvate kinase does not instigate its localization at the nuclear envelope. See Matunis et al. 1998, supra.

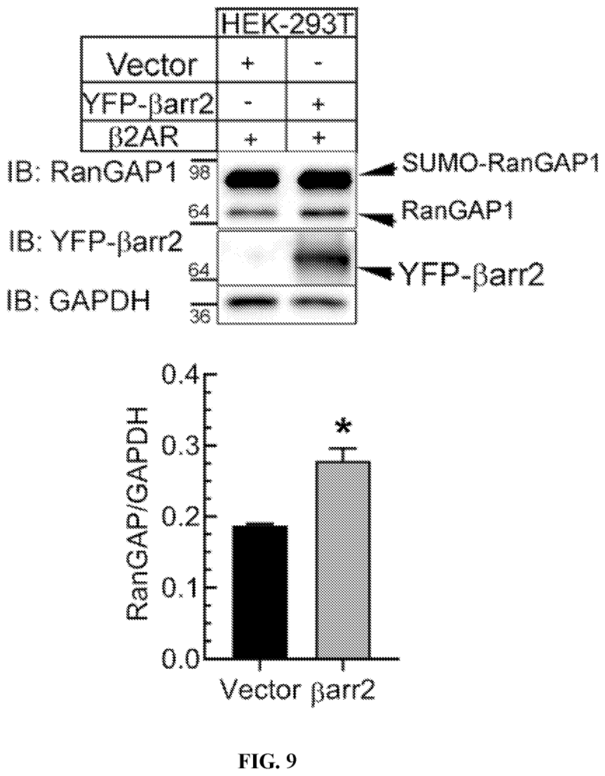

In these experiments, SUMO1 by itself is not localized at the nuclear membrane in HEK-293 cells; only βarrestin2-SUMO1 is localized at the nuclear membrane. Interactome studies have revealed that βarrestin2, but not βarrestin1, can bind RanGAP1 (see Xiao et al. 2007. Proc Natl Acad Sci USA 104:12011-12016); moreover, the localization of RanGAP1 at the nuclear rim overlaps that of the βarrestin2-SUMO1 construct. It is hypothesized that βarrestin2 and its SUMOylation status can regulate RanGAP1 protein association. It is well established that RanGAP1 is expressed in cells predominantly in the SUMOylated state. This is illustrated in immunoblots (FIGS. 9-11), in which the RanGAP1 antibody detects two bands, SUMO-RanGAP1 (approximately 90 kDa) and RanGAP1 (approximately 70 kDa). When βarrestin2 was overexpressed in HEK-293 cells, no dramatic change was detected in the levels of SUMO1-RanGAP1 (90 kDa band, FIG. 9, top panel). On the other hand, in cells with βarrestin2 overexpression, the unmodified RanGAP1 band (approximately 70 kDa) was increased by 50% of the levels in vector transfected cells (FIG. 9, bottom panel).

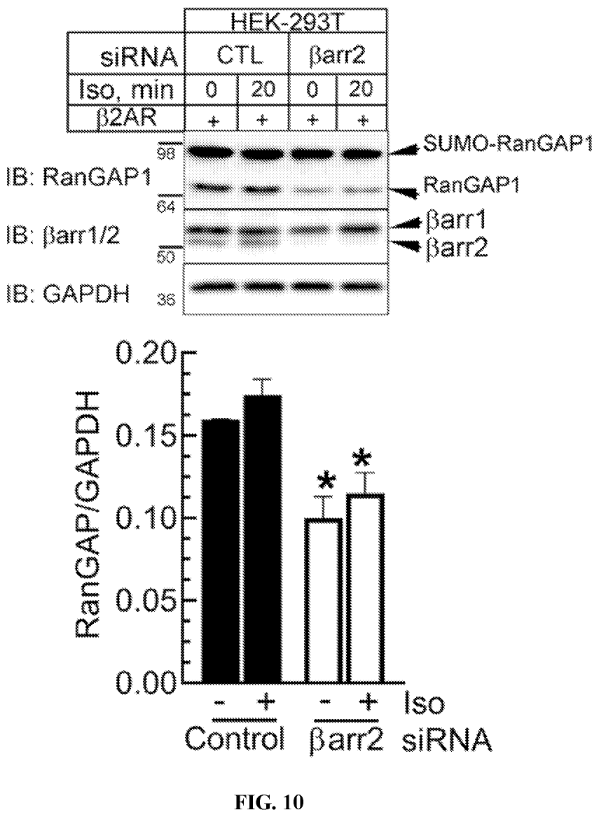

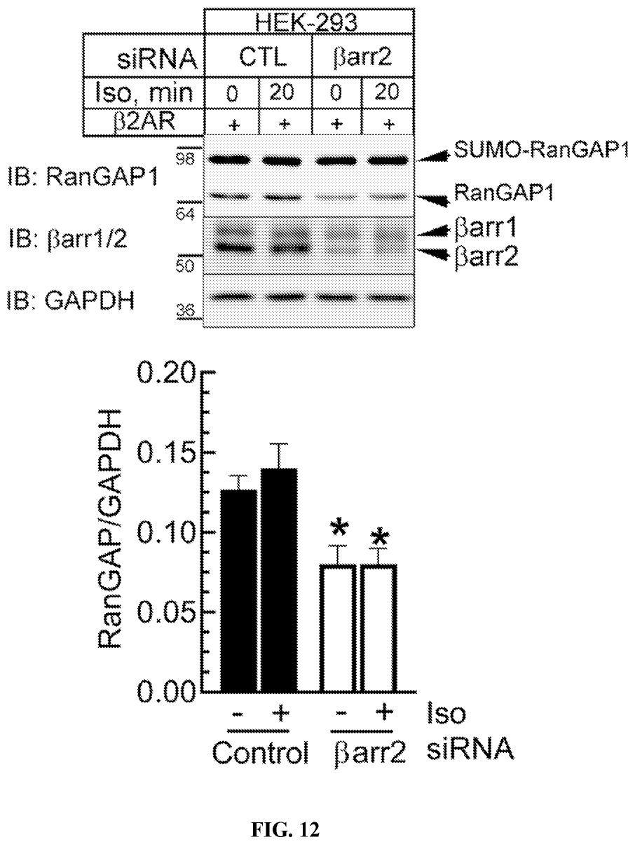

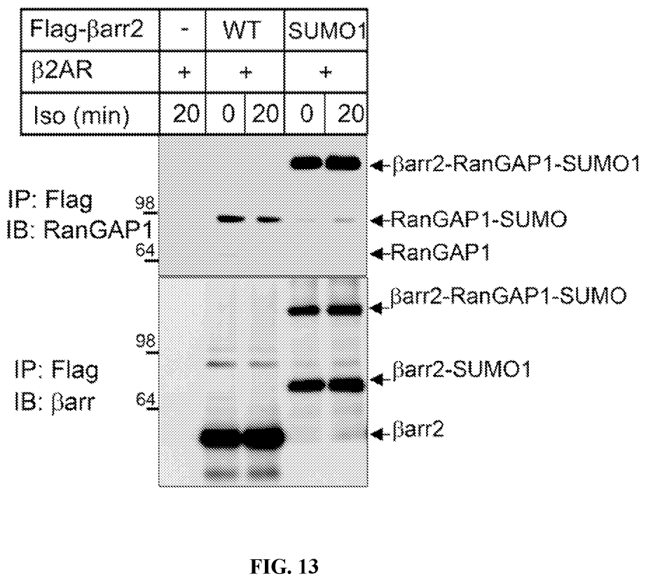

In HEK-293 and HEK-293T cells, which have different levels of endogenous βarrestin2 expression levels, βarrestin2 knockdown by siRNA had no effect on steady-state levels of SUMO-RanGAP1 (FIG. 10, top panel, FIG. 12, and FIG. 13). Nonetheless, a significant decrease in the unmodified RanGAP1 band was observed (FIG. 10, FIG. 12, and FIG. 13). One possibility for the apparent decrease in RanGAP1 in βarrestin2 depleted cells could be an increase in RanGAP1 SUMOylation by Ran binding protein 2 (RanBP2) SUMO E3 ligase (see Werner et al. 2012. Mol Cell 46:287-298), or a decrease in RanGAP1 deSUMOylation by SENP1 that binds βarrestin2. See Xiao et al. 2015, supra. As most of the RanGAP1 in cells exists as the SUMOylated 90 kDa species, and since the RanGAP1-specific antibody detects the 90 kDa band with high avidity, corresponding changes in the SUMOylated RanGAP1 levels were unable to be discerned. On the other hand, these effects of βarrestin2 expression on the expression level of unmodified RanGAP1 could be SUMO1-independent.

To further address the role of βarrestin2-SUMOylation on the sub-cellular localization of RanGAP1, colocalization was observed for endogenously expressed RanGAP1 and overexpressed βarrestin2, or βarrestin2-SUMO1 in HEK-293 cells with or without β₂AR agonist stimulation (data not shown). Minimal colocalization was detected for βarrestin2 and RanGAP1 at the nuclear envelope (one or two punctate structures per cell colocalizing both proteins). However, a dramatic increase in colocalization between βarrestin2-SUMO1 and RanGAP1 was detected, in quiescent cells as well as in cells stimulated with β₂AR agonist. Whether or not βarrestin2-Ub changes the localization of RanGAP1 was also tested. No significant changes in RanGAP1 localization were detected, and nor was any colocalization of βarrestin2-Ub with RanGAP1, suggesting that ubiquitination of βarrestin2 may negatively regulate its interaction with RanGAP1.

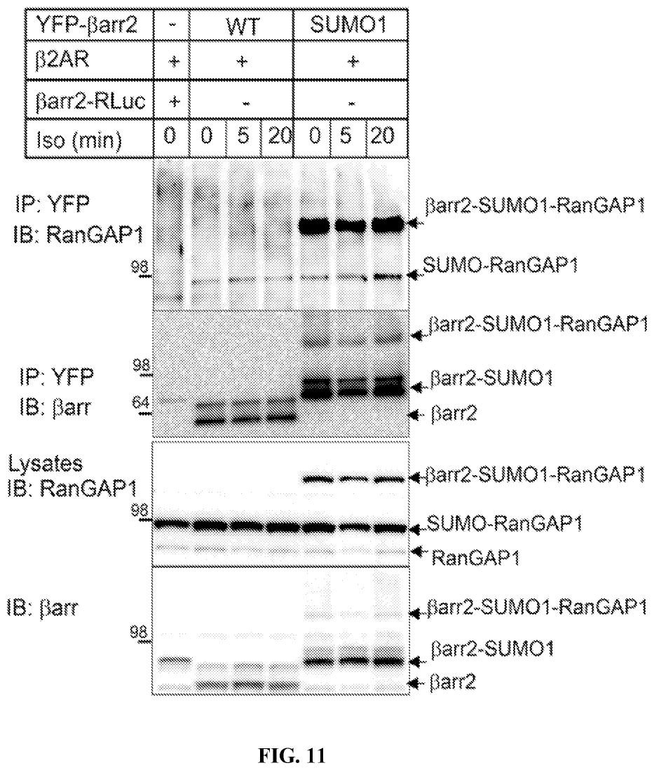

To complement the confocal assessment, coimmunoprecipitation was performed for overexpressed YFP-βarrestin2 or YFP-βarrestin2-SUMO1, and coimmunoprecipitates of YFP proteins were probed for endogenous RanGAP1 (FIG. 11). βarrestin2 that has a non-YFP tag was used as a negative control to ascertain non-specific binding. In these assays, only the SUMOylated form of Ran-GAP1 associated with βarrestin2, and the unmodified RanGAP1 showed weak or no interaction (FIG. 11). βarrestin2-SUMO-RanGAP1 binding was detected in the absence of agonist, which was slightly enhanced by a five-minute agonist-stimulation (1.3±0.4 fold, n=3). βarrestin2-SUMO1 also associated only with SUMO-RanGAP1, but quite strikingly, a macromolecular complex of βarrestin2 and RanGAP1 was detected by immunoblotting with either βarrestin IgG or RanGAP1 IgG (FIG. 11). The interactions between βarrestin2 and endogenous RanGAP1 were further tested by overexpressing Flag-tagged constructs: βarrestin2 and βarrestin2-SUMO1 (FIG. 12 and FIG. 13). Here again, βarrestin2 association with SUMO-RanGAP1 and a macromolecular complex of βarrestin2-SUMO1 and RanGAP1 (FIG. 12 and FIG. 13) were detected. These data suggest that βarrestin2-SUMOylation increases its binding affinity with RanGAP1, and perhaps, SUMOylation of βarrestin2 may be required for its localization at the nuclear membrane and interaction with RanGAP1. Additionally, βarrestin2 might regulate steady state levels of de-SUMOylated RanGAP1, which could proceed concurrently or independently of SUMO1 conjugation of RanGAP1. Taken together, these results suggest a novel role for βarrestin2 in harmonizing conjugation/deconjugation of SUMO1 at the nuclear membrane.

### Example 7: βArrestin1-SUMO1 Fusion Protein Associates with Agonist-Activated β₂AR and Glucagon Receptor with Higher Affinity than βArrestin1

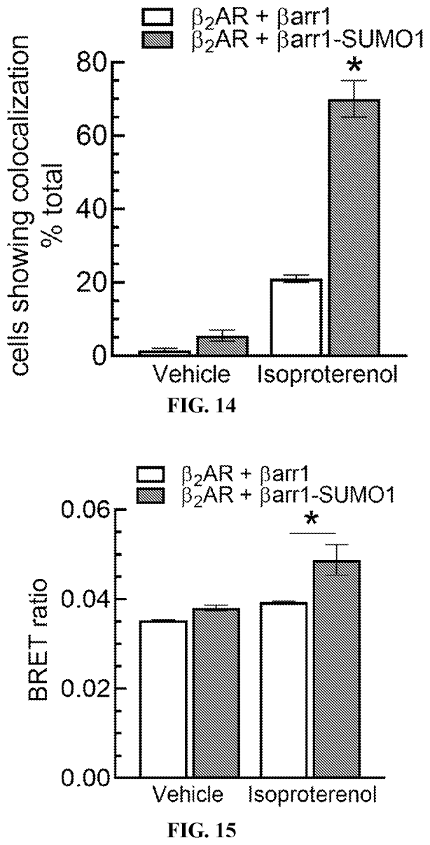

The trafficking of βarrestin1-SUMO1, and βarrestin1, was analyzed in cells with stable expression of the β₂AR and the glucagon receptor (GCGR), similarly to the above analysis of βarrestin-2-SUMO1 and βarrestin2. In quiescent HEK-293 cells, minimal colocalization of βarrestin1 with the β₂AR was observed; upon activation with the agonist isoproterenol for 5 minutes, translocation of βarrestin1 to colocalize with the β₂AR at the plasma membrane was observed (FIG. 14). However, after longer duration of agonist activation, β₂AR and βarrestin1 complexes fall apart with internalization of β₂AR into endosomal vesicles. For βarrestin1-SUMO1, strong colocalization was observed on the endocytic vesicles with β₂AR (FIG. 14). BRET analyses also confirmed that the βarrestin1-SUMO1 fusion protein associates robustly with β₂AR, and more strongly that βarrestin1 alone (FIG. 15).

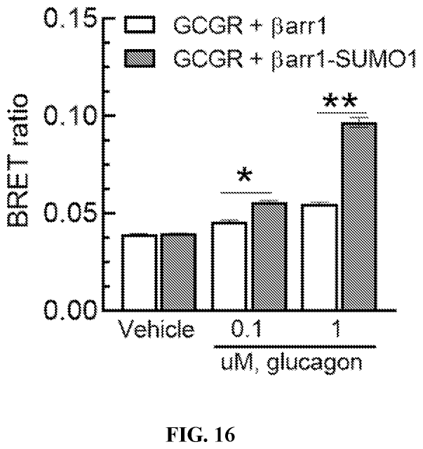

BRET analysis was also used to measure the association between βarrestin1-SUMO1, and βarrestin1, with the glucagon receptor (GCGR). βarrestin1 showed a weak association upon GCGR activation with glucagon, while βarrestin1-SUMO1 showed significantly more binding to the GCGR (FIG. 16).

It is understood that the examples and embodiments described herein are for illustrative purposes only and that various modifications or changes in light thereof will be suggested to persons skilled in the art and are to be included within the spirit and purview of this application and scope of the appended claims. The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group. All publications, patents, and patent applications cited herein and the material for which they are cited are hereby specifically incorporated by reference in their entirety for all purposes.

1. A fusion protein comprising an arrestin polypeptide fused to a ubiquitin-like protein (UBL).

2. The fusion protein of claim 1, wherein the arrestin polypeptide is fused to the UBL protein via a peptide linker.

3. The fusion protein of claim 1, wherein the arrestin polypeptide comprises an amino acid sequence having at least 80% identity to SEQ ID NO:3, SEQ ID NO:4, SEQ ID NO:5, or SEQ ID NO:6.

4. The fusion protein of claim 1, wherein the UBL comprises an amino acid sequence having at least 80% identity to SEQ ID NO:7, SEQ ID NO:8, SEQ ID NO:9, SEQ ID NO:10, SEQ ID NO:11, SEQ ID NO:12, or SEQ ID NO:13.

5. The fusion protein of claim 1, wherein the fusion protein comprises an amino acid sequence having at least 80% identity to SEQ ID NO:1 or SEQ ID NO:2.

6. The fusion protein of claim 1, wherein the fusion protein further comprises a detectable moiety.

7. (canceled)

8. The fusion protein of claim 1, wherein the fusion protein is resistant to de-SUMOylation.

9. The fusion protein of claim 1, wherein the fusion protein displays increased binding to a G protein-coupled receptor (GPCR) upon expression in cells, wherein the increased binding is measured relative to the wild-type form of the arrestin polypeptide.

10. A recombinant nucleic acid encoding the fusion protein of claim 1.

11. A DNA construct comprising a promoter operably linked to the recombinant nucleic acid of claim 10.

12. (canceled)

13. A vector comprising the recombinant nucleic acid of claim 10.

14. A host cell comprising the recombinant nucleic acid of claim 10.

15. (canceled)

16. The host cell of claim 14, wherein the host cell is a mammalian cell.

17-19. (canceled)

20. A method for detecting a protein subcellular localization pattern, the method comprising:
(a) providing a plurality of cells that express the fusion protein of claim 1; and
(b) detecting the subcellular localization pattern of the fusion protein in the plurality of cells.

21. The method of claim 20, wherein the plurality of cells also express a G protein-coupled receptor (GPCR), and wherein the method further comprises detecting the subcellular localization pattern of the GPCR in the plurality of cells.

22. The method of claim 20, wherein the plurality of cells also express a GPCR, and wherein the plurality of cells is treated with an agonist compound that activates the GPCR prior to detecting the subcellular localization pattern of the fusion protein in the plurality of cells.

23. A method for detecting protein-protein interaction of an arrestin protein and a G protein-coupled receptor (GPCR), the method comprising:
(a) providing a plurality of cells that express the fusion protein of claim 1 and a GPCR; and
(b) detecting the protein-protein interaction of the fusion protein with the GPCR in the plurality of cells.

24. The method of claim 23, wherein the plurality of cells is treated with an agonist compound that activates the GPCR prior to detecting the protein-protein interaction of the fusion protein with the GPCR.

25-28. (canceled)

29. The method of claim 21, wherein the GPCR is angiotensin type 1a receptor (AT₁ₐR), β₂ adrenergic receptor (β₂AR), D₂ dopamine receptor (D2R), β₁ adrenergic receptor (β₁AR), D₁ dopamine receptor (D1R), V₂ vasopressin receptor (V₂R), and/or glucagon receptor (GCGR).

30. The method of claim 20, wherein detecting the subcellular localization pattern of the fusion protein is performed by immunostaining, confocal microscopy, bioluminescence resonance energy transfer (BRET), affinity chromatography, and/or immunoprecipitation.

31-32. (canceled)


SEQUENCE LISTING (25 sequences)

SEQ ID NO: 1 - protein, 508 aa, Artificial Sequence
  Synthetic construct
MGEKPGTRVFKKSSPNCKLTVYLGKRDFVDHLDKVDPVDGVVLVDPDYLKDRKVFVTLTCAFRYGREDLDVLGLSFRKDLFIATYQAFPPMPNPPRPPTRLQDRLLKKLGQHAHPFFFTIPQNLPCSVTLQPGPEDTGKACGVDFEIRAFCAKSIEEKSHKRNSVRLIIRKVQFAPETPGPQPSAETTRHFLMSDRRSLHLEASLDKELYYHGEPLNVNVHVTNNSAKTVKKIRVSVRQYADICLFSTAQYKCPVAQLEQDDQVSPSSTFCKVYTITPLLSDNREKRGLALDGQLKHEDTNLASSTIVKEGANKEVLGILVSYRVKVKLVVSRGGDVSVELPFVLMHPKPHDHITLPRPQSAPREIDIPVDTNLIEFDTNYATDDDIVFEDFARLRLKGMKDDDCDDQFCVDSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGG

SEQ ID NO: 2 - protein, 516 aa, Artificial Sequence
  Synthetic construct
MGDKGTRVFKKASPNGKLTVYLGKRDFVDHIDLVDPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIRKVQYAPERPGPQPTAETTRQFLMSDKPLHLEASLDKEIYYHGEPISVNVHVTNNTNKTVKKIKISVRQYADICLFNTAQYKCPVAMEEADDTVAPSSTFCKVYTLTPFLANNREKRGLALDGKLKHEDTNLASSTLLREGANREILGIIVSYKVKVKLVVSRGGLLGDLASSDVAVELPFTLMHPKPKEEPPHREVPESETPVDTNLIELDTNDDDIVFEDFARQRLKGMKDDKDEEDDGTGSPHLNNRVDSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGG

SEQ ID NO: 3 - protein, 410 aa, Rattus norvegicus
MGEKPGTRVFKKSSPNCKLTVYLGKRDFVDHLDKVDPVDGVVLVDPDYLKDRKVFVTLTCAFRYGREDLDVLGLSFRKDLFIATYQAFPPMPNPPRPPTRLQDRLLKKLGQHAHPFFFTIPQNLPCSVTLQPGPEDTGKACGVDFEIRAFCAKSIEEKSHKRNSVRLIIRKVQFAPETPGPQPSAETTRHFLMSDRRSLHLEASLDKELYYHGEPLNVNVHVTNNSAKTVKKIRVSVRQYADICLFSTAQYKCPVAQLEQDDQVSPSSTFCKVYTITPLLSDNREKRGLALDGQLKHEDTNLASSTIVKEGANKEVLGILVSYRVKVKLVVSRGGDVSVELPFVLMHPKPHDHITLPRPQSAPREIDIPVDTNLIEFDTNYATDDDIVFEDFARLRLKGMKDDDCDDQFC

SEQ ID NO: 4 - protein, 418 aa, Rattus norvegicus
MGDKGTRVFKKASPNGKLTVYLGKRDFVDHIDLVDPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIRKVQYAPERPGPQPTAETTRQFLMSDKPLHLEASLDKEIYYHGEPISVNVHVTNNTNKTVKKIKISVRQYADICLFNTAQYKCPVAMEEADDTVAPSSTFCKVYTLTPFLANNREKRGLALDGKLKHEDTNLASSTLLREGANREILGIIVSYKVKVKLVVSRGGLLGDLASSDVAVELPFTLMHPKPKEEPPHREVPESETPVDTNLIELDTNDDDIVFEDFARQRLKGMKDDKDEEDDGTGSPHLNNR

SEQ ID NO: 5 - protein, 409 aa, Homo sapiens
MGEKPGTRVFKKSSPNCKLTVYLGKRDFVDHLDKVDPVDGVVLVDPDYLKDRKVFVTLTCAFRYGREDLDVLGLSFRKDLFIATYQAFPPVPNPPRPPTRLQDRLLRKLGQHAHPFFFTIPQNLPCSVTLQPGPEDTGKACGVDFEIRAFCAKSLEEKSHKRNSVRLVIRKVQFAPEKPGPQPSAETTRHFLMSDRSLHLEASLDKELYYHGEPLNVNVHVTNNSTKTVKKIKVSVRQYADICLFSTAQYKCPVAQLEQDDQVSPSSTFCKVYTITPLLSDNREKRGLALDGKLKHEDTNLASSTIVKEGANKEVLGILVSYRVKVKLVVSRGGDVSVELPFVLMHPKPHDHIPLPRPQSAAPETDVPVDTNLIEFDTNYATDDDIVFEDFARLRLKGMKDDDYDDQLC

SEQ ID NO: 6 - protein, 418 aa, Homo sapiens
MGDKGTRVFKKASPNGKLTVYLGKRDFVDHIDLVDPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIRKVQYAPERPGPQPTAETTRQFLMSDKPLHLEASLDKEIYYHGEPISVNVHVTNNTNKTVKKIKISVRQYADICLFNTAQYKCPVAMEEADDTVAPSSTFCKVYTLTPFLANNREKRGLALDGKLKHEDTNLASSTLLREGANREILGIIVSYKVKVKLVVSRGGLLGDLASSDVAVELPFTLMHPKPKEEPPHREVPENETPVDTNLIELDTNDDDIVFEDFARQRLKGMKDDKEEEEDGTGSPQLNNR

SEQ ID NO: 7 - protein, 96 aa, Homo sapiens
SDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGG

SEQ ID NO: 8 - protein, 71 aa, Homo sapiens
MADEKPKEGVKTENNDHINLKVAGQDGSVVQFKIKRHTPLSKLMKAYCERQLEMEDEDTIDVFQQQTGGVY

SEQ ID NO: 9 - protein, 103 aa, Homo sapiens
MSEEKPKEGVKTENDHINLKVAGQDGSVVQFKIKRHTPLSKLMKAYCERQGLSMRQIRFRFDGQPINETDTPAQLEMEDEDTIDVFQQQTGGVPESSLAGHSF

SEQ ID NO: 10 - protein, 76 aa, Homo sapiens [sequence omitted]

SEQ ID NO: 11 - protein, 165 aa, Homo sapiens
MGWDLTVKMLAGNEFQVSLSSSMSVSELKAQITQKIGVHAFQQRLAVHPSGVALQDRVPLASQGLGPGSTVLLVVDKCDEPLSILVRNNKGRSSTYEVRLTQTVAHLKQQVSGLEGVQDDLFWLTFEGKPLEDQLPLGEYGLKPLSTVFMNLRLRGGGTEPGGRS

SEQ ID NO: 12 - protein, 81 aa, Homo sapiens
MLIKVKTLTGKEIEIDIEPTDKVERIKERVEEKEGIPPQQQRLIYSGKQMNDEKTAADYKILGGSVLHLVLALRGGGGLRQ

SEQ ID NO: 13 - protein, 165 aa, Homo sapiens
MAPNASCLCVHVRSEEWDLMTFDANPYDSVKKIKEHVRSKTKVPVQDQVLLLGSKILKPRRSLSSYGIDKEKTIHLTLKVVKPSDEELPLFLVESGDEAKRHLLQVRRSSSVAQVKAMIETKTGIIPETQIVTCNGKRLEDGKMMADYGIRKGNLLFLASYCIGG

SEQ ID NO: 14 - protein, 12 aa, Artificial Sequence
  Synthetic construct
LDGQLKHEDTNL

SEQ ID NO: 15 - protein, 15 aa, Artificial Sequence
  Synthetic construct
SGSETPGTSESATPE

SEQ ID NO: 16 - protein, 16 aa, Artificial Sequence
  Synthetic construct
SGSETPGTSESATPES

SEQ ID NO: 17 - protein, 15 aa, Artificial Sequence
  Synthetic construct
GGGGSGGGGSGGGGS

SEQ ID NO: 18 - protein, 50 aa, Artificial Sequence
  Synthetic construct
GGGGSGGGGSGGGGSGGGGSGGGGSGGGGSGGGGSGGGGSGGGGSGGGGS

SEQ ID NO: 19 - protein, 100 aa, Artificial Sequence
  Synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 20 - protein, 17 aa, Artificial Sequence
  Synthetic construct
AEAAAKEAAAKEAAAKA

SEQ ID NO: 21 - protein, 52 aa, Artificial Sequence
  Synthetic construct
AEAAAKEAAAKEAAAKEAAAKEAAAKEAAAKEAAAKEAAAKEAAAKEAAAKA

SEQ ID NO: 22 - protein, 6 aa, Artificial Sequence
  Synthetic construct
HHHHHH

SEQ ID NO: 23 - protein, 9 aa, Artificial Sequence
  Synthetic construct
YPYDVPDYA

SEQ ID NO: 24 - protein, 8 aa, Artificial Sequence
  Synthetic construct
DYKDDDDK

SEQ ID NO: 25 - protein, 10 aa, Artificial Sequence
  Synthetic construct
EQKLISEEDL